(19) United States
(12) Patent Application Publication
(10) Pub. No.: US 2002/0115604 A1
(43) Pub. Date: Aug. 22, 2002

(54) HCaRG, a novel calcium-regulated gene coding for a nuclear protein

(76) Inventors: Johanne Tremblay, Quebec (CA); Pavel Hamet, Quebec (CA); Richard Lewanczuk, Alberta (CA); Francis Grossard, Quebec (CA); Nicolas Solban, Quebec (CA)

Correspondence Address: BANNER & WITCOFF, 1001 G STREET N W, SUITE 1100, WASHINGTON, DC, 20001, US

(21) Appl. No.: 09/904,568

(22) Filed: Jul. 16, 2001

Related U.S. Application Data
(63) Continuation-in-part of application No. 09/223,796, filed on Dec. 31, 1998, now abandoned.
Application No. 09/223,796 is a continuation-in-part of application No. 08/667,495, filed on Jun. 21, 1996, now abandoned.

(30) Foreign Application Priority Data
Jul. 14, 2000 (CA) 2,312,266

(51) Int. Cl. A61K 38/17; A61K 48/00; C12P 21/02; C12N 5/06; C12N 9/64; C07H 21/04

(52) U.S. Cl. 514/12; 514/44; 435/69.1; 435/226; 435/325; 435/320.1; 536/23.2

(57) ABSTRACT

This invention relates to a novel gene that shows tissue specific expression and increased expression in a low calcium concentration medium. Low renin hypertension is characterized by decreased levels of serum ionized calcium in the presence of increased levels of parathyroid hormone. It is hypothesized that hypertensive factor(s) are co-secreted with PTH in SHR, a model of low renin hypertension, the parathyroid hypertensive factor being one of them. As a negative calcium balance is present in spontaneously hypertensive rats (SHR), we searched for gene(s) involved in this dysregulation. A cDNA library was constructed from the SHR parathyroid gland which is a key regulator of serum ionized calcium. From 7 overlapping DNA fragments, a 1100-bp novel cDNA containing an open reading frame of 224 codons was reconstituted. This novel gene, named HCaRG (Hypertension-related, Calcium-regulated Gene), was negatively regulated by extracellular calcium concentration and its basal mRNA levels were higher in hypertensive animals. The deduced protein showed no transmembrane domain, 67% a helix content, a mutated calcium-binding site (EF-hand motif), 4 putative ‘leucine zipper’ motifs and a nuclear receptor-binding domain. At the subcellular level, HCaRG had a nuclear localization. We cloned the human homolog of this gene. Sequence comparison revealed 80% homology between rats and humans at the nucleotide and amino acid sequences. Tissue distribution showed a preponderance in the heart, stomach, jejunum, kidney (tubular fraction), liver and adrenal gland (mainly in the medulla). HCaRG mRNA was significantly more expressed in adult than in fetal organs, and its levels were decreased in tumors and cancerous cell lines. We observed that after 60-min ischemia followed by reperfusion, HCaRG mRNA declined rapidly in contrast with an increase in c-myc mRNA. Its levels then rose steadily to exceed baseline at 48 h of reperfusion. HEK293 cells stably transfected with HCaRG exhibited much lower proliferation, as shown by cell count and 3 H-thymidine incorporation. Taken together, our results suggest that HCaRG is a nuclear protein potentially involved in the control of cell proliferation.

### FIELD OF THE INVENTION

The present invention relates to a novel gene that shows tissue specific expression and increased expression in a low calcium concentration medium and in hypertensive animals, and which is potentially involved in the regulation of cell proliferation.

### BACKGROUND OF THE INVENTION

Calcium ion is an essential element of life with distinct extracellular and intracellular roles- Extracellular functions of calcium include its role in blood clotting, intercellular adhesion, bone metabolism, maintenance of plasma membrane integrity whereas its intracellular roles include protein secretion, cellular contraction and division. The free extracellular calcium concentration is maintained within a narrow range (˜1 to 1.3 mM) and that of intracellular calcium is in the order of 100 nM; 10,000 fold lower than the extracellular free calcium concentration.

The first priority of the extracellular calcium homeostatic system is to maintain a normal extracellular ionized calcium concentration. This component represents approximately 45% of the total circulating calcium concentration- Another 45% of total circulating calcium is bound to proteins (primarily albumin) and about 10% is Pound to small organic anion. Therefore, ionized calcium concentration in plasma is maintained within a very narrow range. The major players maintaining extracellular calcium homeostasis are calciotropic hormones, parathyroid hormone (PTH), 1,25 dihydroxyvitamin D, calcitonin and calcium itself. Indeed, extracellular calcium regulates its own concentration as an extracellular messenger by acting on cells involved in the control of extracellular calcium homeostasis such as parathyroid, bone, intestine and kidney cells (56). For example, parathyroid cells are key sensors of extracellular calcium in vertebrates responding with increases in PTH secretion when there is a decrease in calcemia while high calcemia stimulates hormonal release of calcitonin from C cells of the thyroid gland.

Cells of the parathyroid gland possess such a calcium sensor (6). Even slight reductions in extracellular ionized calcium concentration (in the order of 1-2% or less) elicit prompt increases in the rate of PTH secretion and mRNA levels Renal responses to the increase in circulating levels of PTH relevant to mineral ion metabolism include phosphaturia and enhanced distal tubular reabsorption of calcium The most rapid changes in calcium handling by the target tissues of PTH take place in the kidneys and skeleton.

The parathyroid gland is particularly well positioned to respond to hypocalcemic stresses. The parathyroid cells (and probably few other cell types) are capable of sensing the changes in the extracellular calcium concentration. The process of calcium sensing (that is a capacity to recognize and respond to physiologically meaningful changes in extracellular calcium), differs from simple calcium dependence. A parathyroid calcium receptor has been recently characterized. It is present on the cell surface and interacts not only with calcium but also with a variety of other divalent cations as well as with polycations. The receptor has probably at least two binding sites that confer positive cooperativity to it. The putative calcium receptor is linked to several intracellular second messenger systems via quanylyl nucleotide regulatory G proteins and activate a phosphoinositide specific phospholipase C leading to accumulation of inositol 1,4,5 trisphosphate (IP3) and diacylglycerol (1-5). Such a receptor is also found in the proximal tubular cells consistent with a regulation of tubular function through a mechanism similar to that in parathyroid cells Hypocalcemia promotes parathyroid cellular hypertrophy and increases levels of the mRNA for PTH. 1,25 dihydroxyvitamin D has a clear inhibitory effects on parathyroid cellular proliferation

Historically, research on the parathyroid gland has focused on the chemistry, regulation, synthesis and secretion of PTH. There is growing interest in other calcium-regulating proteins of this gland that are also negatively regulated by extracellular calcium, such as chromogranin A and Secretory Protein-I (7), as well as a hypertensive factor of parathyroid origin (PHF) (8,9). This hypertensive factor of parathyroid origin has been recently documented with similarities to an intracellular calmodulin-PDE activator, described in hypertensive tissues and organs (57,58). This factor increases blood pressure when injected into anesthetized rats and has been shown to potentiate the action of pressor agents (norepinephrine) on the contraction of vascular smooth muscle (59).

Diseases associated with hypertension include arteriosclerosis, hypertensive renal failure, stroke, heart failure and myocardial infarction, to name a few. While methods to treat hypertension are available, the etiology of hypertension, for the most part, remains unknown.

A number of persons have attempted to purity the active component of parathyroid hypertensive factor in an attempt to improve methods of treating patients with diseases which involve extracellular calcium elevation, such as hypertension. In one patent application, PIT 93US6626, the inventors describe a purified and isolated parathyroid hypertensive factor component including a polypeptide linked to a phospholipid. This component produces a delayed onset of an increase in blood pressure of a normotensive rat to which it is administered. The increase in blood pressure is said to temporarily correlate with an increase in extracellular calcium uptake by vascular smooth muscle. However, this factor, when highly purified, is not greatly increased in hypertensive states.

Similarly, other hypertensive factors derived from parathyroid gland are described in other patent applications, such as Japanese patent application 4134098 and PCT 90US1577. The factors are obtained by culturing, dialysing, ultrafiltering, refrigerating drying plasma component and separating the active fraction by gel filtration column chromatography. Again, these factors are not greatly increased in hypertensive states.

Despite the work that has been done in the area of hypertensive factors, a need still exists to identity a mammalian gene which is increased in hypertensive states. This gene could be used (1) to treat diseases related to modulation in calcium levels, (2) to screen pharmaceutical components which are effective in treating diseases related to modulation in calcium levels, or (3) for the diagnosis of the presence of diseases related to modulation in calcium levels. Diseases related to modulation in calcium levels include hypertension, hyperthyroidism, osteoporosis, osteopetrosis, heart failure. Insulin dependent and independent diabetes, cancer (including breast, thyroid, colon, kidney and leukaemia), disorders of the central nervous system including stroke, artherosclerosis, gastrointestinal diseases, inflammatory bowel disease and asthma.

In particular, arterial hypertension is associated with numerous disturbances of calcium metabolism manifested not only in humans but also in genetic as well as acquired models of hypertension (10-14). Disturbances in renal and intestinal handling of calcium in hypertension have been reported by several investigators (15). Urinary calcium has generally been shown to be increased (so called urinary leak) and intestinal calcium absorption diminished in genetically hypertensive or spontaneously hypertensive rats (SHR) (15,16). Cytoplasmic free calcium concentration has most often been found to be elevated in circulating platelets, lymphocytes, erythrocytes, and vascular smooth muscle cells (VSMC) from hypertensive animals and humans (for review see (17)). In SHR as well as in low-renin hypertensive patients, there seems to be an inverse relationship between extracellular and intracellular calcium (18) It has been hypothesized that certain genetic abnormalities might be responsible for the link between some foams of hypertension, calcium homeostasis and the parathyroid gland. To identify new genes that might be abnormally regulated by extracellular calcium in the parathyroid gland of genetically hypertensive rats, the present inventors prepared a cDNA library from the parathyroids of SHR. In this study, the present inventors describe the isolation and characterization of a novel gene, designated HCaRG (for Hypertension-related, Calcium-regulated Gene), negatively regulated by extracellular calcium with higher mRNA levels in SHR. HCaRG is a nuclear protein with putative ‘leucine zipper’ motifs and is potentially involved in the regulation of cell proliferation.

### SUMMARY OF THE INVENTION

The present inventors have identified a new gene expressed in the parathyroid gland. The expression of this gene is regulated in a way similar to that of PTH, that is hypocalcemia increases its mRNA levels. Experiments involved spontaneously hypertensive rats (SHR), models of low renin hypertension, and normotensive counterparts Wistar-Kyoto (WKY). The expression of this novel gene was higher in the SHR parathyroid cells than in cells from WKY. In situ hybridization studies showed that this gene has a specific pattern of expression It is highly expressed in the tubular fraction of the renal cortex, in the medulla and the inner part of the adrenal cortex, in the intestine, in the heart and in the brain.

Therefore, the present invention relates to a nucleic acid molecule isolated from parathyroid of a mammal and whose expression is regulated by extracellular calcium concentration. In one case, the mammal is a human and the molecule encodes the amino acid sequence set out in FIG. 4 (bottom lines). In another case, the mammal is a rat and the molecule encodes the amino acid sequence set out in FIG. 4 (bottom lines) The invention includes a nucleotide molecule of a human, and having a homology of 60% or greater to all or part of the sequence set out in FIG. 1. The molecule may have a 60% or greater homology to the translated portion of the sequence.

The invention also includes a purified and isolated protein (HCaRG) encoded by the nucleic acid molecule of this invention. Mimetics of and antibodies to this protein are included within this invention as are proteins having a homology of 60% or greater to the proteins encoded by the nucleic acid molecules of this invention.

The invention also suggests that HCaRG is a nuclear protein potentially involved in the control of cell proliferation, since HCaRG mRNA was significantly more expressed in adult than in fetal organs, and its levels were decreased in tumors and cancerous cell lines. In addition, the present inventors observed that after 60 min ischemia followed by reperfusion, HCaRG mRNA declined rapidly in contrast with an increase in c-myc mRNA. Its levels then rose steadily to exceed baseline at 48 h of reperfusion. As an evidence that HCaRG can be used to treat a disease inhibing calcium, HEK293 cells stably transfected with HCaRG and overexpressing the same, exhibited much lower proliferation, as shown by cell count and 3 H-thymidine incorporation

A pharmaceutical composition of this invention would include at least a portion of the protein encoded by the nucleic acid molecules of this invention or in the alternative, a pharmaceutical composition could include a nucleic acid molecule of this invention, or a portion thereof, for use in gene therapy. The composition could be used to treat a patient suffering from a condition caused try the abnormal intracellular or extracellular modulation of calcium or abnormal proliferative disorders comprising administering an effective amount of a sense molecule hybridizing with the nucleic acid of FIG. 1, for example, (to upregulate the molecule's expression) or an antisense molecule, for example, (to downregulate the molecule's expression) to the patient.

The molecule could be delivered as part of a recombinant vehicle, or in liposomes, for example. In one case, the molecule would include a sense or an antisense sequence to all or part of the nucleic acid sequence of a human gene sequence encoding the protein set out in FIG. 4 The sense sequence would enhance the effect of the protein which sequence is set out in FIG. 4. The antisense sequence would, on the contrary, suppress the effect of the same.

Included within this invention is a kit for the detection of a disease, disorder or abnormal physical state caused by abnormal modulation of calcium levels in a patient. The kit could include, as a target or as a marker, all or part of the nucleic acid molecule of this invention, for example, the sequence of a human gene encoding the protein set out in FIG. 4. In another case, a kit could include as a marker or as a target all or part of a protein encoded by a nucleic acid molecule of this invention, a mimetic of such a protein or an antibody to such a protein. The kit could be used to help diagnose hypertension, hyperthyroidism, osteoporosis, heart failure, insulin dependent and independent diabetes, disorders of the central nervous system including stroke, cancer (including prostate, ovary, breast, thyroid, colon, kidney and leukaemia), artherosclerosis, gastrointestinal diseases, inflammatory bowel disease and asthma. Once diagnosed, patients may wish to regulate extracellular calcium uptake by increasing dietary calcium levels or taking calcium supplements.

Also included within this invention is the use of the pharmaceutical compositions of this invention to treat a patient having a disease, disorder or abnormal physical state related to abnormal intracellular or extracellular calcium levels Also included is the use of the protein of this invention or the mimetics of such protein to screen for inhibitors to such protein.

A method for assaying for abnormal intracellular or extracellular calcium levels would include (a) reacting a sample of a patient with a nucleic acid molecule of this invention, or a portion thereof, under conditions where the sample and the molecule, or a portion thereof, are capable of forming a complex; (b) assaying for complexes, free molecule, or a portion thereof, and (c) comparing with a control. In one case, the molecule is a sense or an antisense sequence to all or part of the human gene sequence encoding a protein as set out in FIG. 4.

In another assay for abnormal intracellular or extracellular calcium levels, the assay includes (a) reacting a sample of a patient with a protein of this invention, or a portion or a mimetic thereof, or an antibody thereto, under conditions where the sample and the protein, or a portion or a mimetic thereof, or an antibody thereto, are capable of forming a complex; (b) assaying for complexes, free protein, or a portion or a mimetic thereof, or an antibody thereto, and (c) comparing with a control.

A method for differenciating normal cells and cells of a tissue exhibiting an abnormal intra-cellular or extra-cellular calcium level (such as “diseased tissue” includes but is not limited to cancer cells), is also within the scope of this invention. This method involves a step of contacting a diseased tissue with a detectable ligand which binds to HCaRG protein or nucleic acids. Examples of ligands are hybridizing probes, antagonists or antibodies. The binding of the ligand to a diseased tissue would provide boundaries that can be visualized by a therapist or surgeon, for differentiating normal tissues, not to be treated or excised, from diseased tissues to be treated (by radiotherapy or chemotherapy, for example) or excised (by surgery).

In yet another assay for screening for efficacy of products modulating (enhancing or inhibiting) abnormal calcium levels, the assay includes (a) reacting a sample of a patient with a protein of this invention, or a portion or a mimetic thereof, or an antibody thereto, under conditions where the sample and the protein, or a portion or a mimetic thereof, or an antibody thereto, are capable of forming a complex; (b) assaying for complexes, free protein, or a portion or a mimetic thereof, or an antibody thereto, and (c) comparing with a control.

Furthermore, another assay for screening for efficacy of a product for modulating (enhancing or inhibiting) abnormal intracellular or extracellular calcium levels could include (a) reacting the product with a protein of this invention, or a portion or a mimetic thereof, or an antibody thereto, under conditions where the product and the protein, or a portion or a mimetic thereof, or an antibody thereto, are capable of forming a complex; (b) assaying for complexes, free protein, or a portion or a mimetic thereof, or an antibody thereto, and (c) comparing with a control.

This invention includes a method for screening for efficacy of a product for use in modulating (enhancing or inhibiting) abnormal intracellular or extracellular calcium levels, the assay includes (a) reacting the product with a nucleic acid molecule of this invention, or a portion thereof, under conditions where the product and the molecule, or a portion thereof, are capable of forming a complex; (b) assaying for complexes, free molecule, or a portion thereof, and (c) comparing with a control.

Other objects, features and advantages of the present invention will become apparent from the following detailed description. It should be understood, however, that the detailed description and the specific examples while indicating preferred embodiments of the invention are given by way of example only, since various changes and modifications within the spirit and scope of the invention will become apparent to those skilled in the art from this detailed description.

### DESCRIPTION OF THE FIGURES

The invention will now be described in relation to the figures in which:

FIG. 1: cDNA cloning of HCaRG.

A. Reconstitution scheme of HCaRG cDNA. Overlapping fragments leading to the reconstitution of rat HCaRG 1100-bp cDNA are shown. cDNA fragments were initially obtained using 5′-RACE and 3′-RACE strategies as well as by screening a SHR parathyroid cDNA library. The first cDNA fragment was by 3′-RACE (3r 290) This initial fragment served to screen the SHR parathyroid cDNA library. Fragments HCaRG 2c-t3 t 2c-t7, HCaRG 825. HCaRG 10-ic, and HCaRG 10-174 were isolated from the cDNA library. Fragments 5r 285 and 5r 260 were obtained by 5′-RACE. This reconstitution was confirmed by sequencing a 860-bp PCR product with nested primers in 5r 260 and HCaRG 825 and containing the complete open reading frame.

B. Nucleotide and deduced amino acid sequences of HCaRG. The translation initiation start site codon is at position 1 and the termination codon is at position 675. The deduced amino acids are indicated below the nucleotide sequence. The localization of a 482-bp intron is indicated at position -52 by a triangle.

FIG. 2. Identification of i novel gene negatively regulated by extracellular calcium.

A. Northern blot analysis of Poly A RNA isolated from parathyroid cells (PTC). HCaRG mRNA appears as a doublet of approximately 1.2 and 1.4 kb. The positions of ribosomal RNAs and GAPDH transcript are indicted.

B. PTC extracted from normotensive rats (WKY) (from passages 8 to 10) were incubated in low (0.3 mM) or normal (2 mM) calcium-containing medium for 2 and 48 h. Total RNA was extracted and analysed by RT-PCR as described in the Experimental Procedures section. Incubation of PTC for 2 h in 0.3 mM (L) calcium significantly increased HCaRG mRNA compared to 2 MM (N) calcium; this increase lasted up to 48 h.

C. Significantly higher basal HCaRG levels were found in PTC from hypertensive rats compared to the normotensive rat strain WKY (left panel) P. This was confirmed with RNA (right panel) and proteins extracted directly from the kidneys of SHR and BN.lx, another normotensive strain. The figure represents the mean ±S.E.M. of 2 independent experiments performed in duplicate. ** indicates p<0.02, * indicates p<0.05 as evaluated by the unpaired t-test.

FIG. 3: In vitro translation of HCaRG cDNA. cDNA was cloned into pSP72 vector and used for coupled transcription/translation in the presence of 35 S-methionine. Lane 1: molecular weight markers; lane 2: translation products of the control luciferase gene; lane 3: translation products without the insert, lane 4: translation product from HCaRG cDNA; lane 5: translation products of HCaRG cDNA The proteins were separated by 15% PAGE in the presence (lanes 1 to 4) or absence (lane 5) of b-mercaptoethanol. Transcription/translation of HCaRG cDNA yields a protein of 27 kDa (lane 4). In the absence of b-mercaptoethanol, a product of 43 kDa was also observed (lane 5), suggesting intramolecular or intermolecular disulfide bridges and the formation of homodimers or heterocimers with other protein(s) present in the lysate.

FIG. 4: Sequence comparison between human HCaRG and rat HCaRG The deduced amino acid sequences of rat HCaRG (rHCaRG) and of human HCaRG (hHCaRG) are aligned. Identical amino acids are boxed while homologous amino acids are shaded. We calculated 80% homology between these 2 sequences. Analysis revealed homology to the EF-hand motif, with 8 out of the 10 most conserved amino acids (dashed box). Further analysis using the PROSEARCH database revealed 4 overlapping putative ‘leucine zipper’ consensus motifs (underlined). We also identified a nuclear receptor-binding domain (bold and italics).

FIG. 5; Subcellular localisaton of HCaRG in cultured cells. COS-7 cells were transfected with GFP-HCaRG. 24 h later, the cells were fixed and observed. Cells transfected with pEGFP vector alone show diffuse fluorescence (A) while cells transfected with pEGFP-HCaRG present nuclear fluorescence (B). Nuclear localization was confirmed by immunofluorescence on COS7 cells transfected with pcDNA1/Neo-HCaRG (C), and by electron microscopy (D) on pituitary.

FIG. 6: Tissue distribution of HCaRG mRNA.

A. Comparison of HCaRG expression in fetal versus adult human organs. HCaRG mRNA is expressed less in all fetal tissues compared, particularly in the heart. Kidney and liver ( adult; fetal).

B. Northern blot containing 2 μg of polyA+RNA from fetal and adult human hearts. HCaRG is more expressed in all regions of the adult heart (L: left, R: right).

C. Comparison of HCaRG expression in adult human organs versus cancerous cell lines. HCaRG mRNA is expressed less in most cancerous cell lines compared. Lymphocyte (normal; Burkitt's lymphoma Raji; Burkitt's lymphoma Daudi). Leukocyte ( normal; leukemia HL-60 leukemia K-562; leukemia MOLT4). Rectum (normal; colorectal adenocarcinoma SW480). Lung (normal; lung carcinoma A549).

D. Northern blot containing 20 μg of total RNA isolated from 3 different human tumours (T) and normal tissue (N) excised at the same operational site. HCaRG expression is decreased in brain, kidney and liver tumours.

FIG. 7: In situ hybridization of HCaRG mRNA in the kidney and adrenal, in situ hybridization of HCaRG mRNA in the rat adrenal shows specific detection in the zona fasciculata and medulla. Specific hybridization in the kidney is restricted to proximal tubules, contrasting with virtual absence in the glomeruli (G). (Upper panels: antisense probe, lower panels; sense probe).

FIG. 8: Analysis of kidney mRNA of HCaRG and c-myc obtained after ischemia and various periods of reperfusion.

A. Dot blot of total RNA taken from the medulla of kidneys which underwent 60-min ischemia and reperfusion for various time periods (full lines) or from contralateral control Kidneys (dotted lines) HCaRG mRNA declined rapidly to its lowest levels at 3 h and 6 h of reperfusion. It then increased steadily to exceed baseline at 48 h of reperfusion. In contrast, c-myc mRNA levels rose dramatically by 12 h and returned below HCaRG mRNA levels at 48 h of reperfusion.

B. Representative northern blots of HCaRG and c-myc mRNA from the cortex of kidneys which underwent 60-min ischemia and 3 h, 6 h, 12 h, 24 h or 48 h (HCaRG) or 12 h or 24 h (c-myc) of reperfusion (VR) or from contralateral control kidneys (c).

FIG. 9: Characterization of stable call lines.

A. HEK293 cells transfected with pcDNA1/Neo or pcDNA1/Neo rat HCaRG were examined for expression of rat HCaRG by northern blot using rat HCaRG as a probe. Rat HCaRG was undetectable in cells transfected with the empty vector while different levels of expression were observed in cells transfected with the vector expressing HCaRG.

B. The levels of ectopic expression were determined by densitometric measurement and normalized to GAPDH.

FIG. 10: HCaRG expression inhibits cell proliferation.

A Stable clones Neo1, Neo6. Neo Poly, HCaRG8, HCaRG9, and HCaRG Poly were plated at low density. For each time point, triplicate plates were counted, and average cell number was recorded. The level of DNA synthesis was monitored by measuring [³H] thymidine incorporation (B). Representative experiment performed in triplicate

FIG. 11: Localization of HCaRG on rat chromosome 7.

A HCaRG Bgl 11polymorphism was used as marker on genomic DNA from SHR and BN.1× rat inbred strains with multiple well characterized SDPs. HCaRG cosegregated with D7Cebrpl87s3/D7Cebr77si of rat chromosome 7 in 31 out of 33 stains. Two recombinations mapped the HCaRG between Cyp 11β₂ and Myc genes. cM represents distance in centimorgans on rat chromosome 7. On the right side, a possible linkage position of human homologous gene is depicted as based on conserved linkages on rat chromosome 7 and human chromosome 8.

### DETAILED DESCRIPTION OF THE INVENTION

The cloning of a novel extracellular calcium-responsive gene (HCaRG) in the rat parathyroid gland from SHR is described here. HCaRG mRNA and protein levels were higher in cultured PTC and in several organs of SHR, compared to their normotensive counterparts. They were negatively regulated by extracellular calcium, i.e. lowering extracellular calcium led to increases in HCaRG mRNA The identification of an extracellular calcium-sensing receptor from the parathyroid gland has provided novel insights into the mechanisms of direct action of extracellular calcium on several cell types. The calcium sensor has also been localized in the cerebral cortex and cerebellum, in the tubular region of the kidney cortex, the thyroid, adrenal medulla, lung, and blood vessels (1,32,33). As shown here, HCaRG mRNA levels are also detected in several of these tissues. The calcium receptor is a member of the superfamily of G protein-coupled receptors activating phospholipase C (34,35). In the parathyroid gland, it is a key mediator of inhibition of PTH expression by high calcium (36). The calcium sensor has been shown, in the kidney, to be directly related to inhibition of tubular reabsorption of calcium and magnesium in the thick ascending loop (for review, see (34)). In PTC cultures prepared from human or bovine parathyroids, low extracellular calcium (0.3 mM) has been demonstrated to increase PTH secretion and mRNA levels whereas augmentation of calcium in the incubation medium reduces PTH mRNA. Similar regulation was observed for PHF in rat parathyroid cells (9) The present inventors show here that HCaRG expression is regulated in a manner similar to PTH and PHF in PTC isolated from the rat.

To date, very few extracellular calcium-negative responsive genes have been cloned. Parathormone was the first gene described to possess a negative calcium-responsive element (nCARE) in its 5′flanking region (37). Several types of nCARE have been reported: Type 2 is a regulatory element consisting of a palindromic core sequence and several upstream T nucleotides originally described in the PTH gene. Its transcriptional inhibitory activity is orientation-specific. The nCARE core is present in an Alu-repeat in 111 copies in the human genome, suggesting the possibility that other genes may possess functional nCARE (38). With the properties described in the present study, HCaRG may be one of them.

HCaRG is not only expressed in the parathyroid gland but also in most organs tested, although at highly variable levels. Elevated HCaRG levels have been noted consistently in the tissues of genetically hypertensive animals, suggesting abnormalities of HCaRG regulation in several organs of SHR that could be due to either: 1) decreased extracellular calcium levels; 2) an abnormal response to extracellular calcium; 3) abnormal transcription/stability of HCaRG mRNA in hypertensive rats, or 4) a combination of these. A state of negative calcium balance has been described in SHR that could support the first possibility. On the other hand, 2-fold higher HCaRG mRNA levels were observed in PTC from SHR than from WKY at normal calcium concentration (FIG. 2C). Thus, the modest reduction of calcemia in hypertension will not be the sole explanation of increased levels, suggesting increased expression or decreased degradation of this gene product in hypertension.

No homologous protein sequence to the HCaRG open reading frame was found in the SWISSPROTEIN database. The HCaRG coding sequence contains 1 consensus motif known as the EF-hand or HLH Ca motif (FIG. 3 dashed box). This motif generally consists of a 12-residue, Ca-binding loop flanked by 2 a-helices Eight of the 10 most conserved amino acids are present in HCaRG protein. Usually, the basic structural/functional unit consists of a pair of calcium binding sites rather than a single HLH motif The HCaRG coding sequence contains only 1 EF like motif but it is possible that its high a-helix content favours coiled-coil interactions and dimerization of the protein. Pairing of the 2 EF-hand motifs may enhance ts calcium function. Hodges and collaborators (39,40) have demonstrated that domain III of troponin C (a synthetic 34-residue calcium-binding domain) can form a symmetric 2-site homodimer in a head-to-tail arrangement in the presence of calcium (41). Similarly, a 39-residue proteolytic fragment containing calcium-binding site IV of troponin C was shown to form a dimer (42). These studies and others (43-45) have demonstrated that dimerization of single HLH structures controls calcium affinity and that even homodimers can bind 2 calcium molecules with positive cooperativity (40). Hydrophobic interactions at the interface between calcium-binding sites appear to stabilize the calcium domains. The present inventors' in vitro translation studies showed the appearance of a protein band of about 43 kDa under non-reducing conditions. HCaRG protein might form reductant-sensitive, non-covalent homodimers compatible with its putative high a-helix content, but the existence of a functional calcium domain in HCaRG protein remains to be established. Several characteristics of HCaRG are similar to those of S100A2 protein, a calcium binding protein of the EF-hand type that is preferentially expressed in the nucleus of normal cells but down-regulated in tumors (44). As with HCaRG, S100A2 expression is down-regulated by calcium (46,47).

The present inventors also cloned the human homolog of HCaRG from a VSMC cDNA library, using a 437-bp fragment of rat HCaRG as a probe. The coding sequence was found to be 80% homologous to the rat sequence and to contain the putative EF-hand domain- A restriction fragment length polymorphism permitted the present inventors to localize the HCaRG locus on chromosome 7 of rats (FIG. 11). The gene was assigned within a 4.4-cM region on the long arm of chromosome 7 between Mit 3 and Mit 4 genes. By analogy, the present inventors suggested the assignment of HCaRG on human chromosome 8q21-24. In a recent search of HCaRG homologous sequences in Genbank, homologies were found with 3 chromosome 8 clones containing ZFP7. It was, therefore, possible to localize HCaRG on chromosome 8q24.3, confirming the present inventors' initial assignment (FIG. 11). This region contains loci involved in several bone diseases, including osteopetrosis and multiple exostosis and several human neoplasms (48,49).

Many DNA-binding proteins utilize zinc-containing motifs to bind DNA. Other classes of DNA binding proteins have a DNA-recognition domain at their N terminus that dimerizes to form a 2-chain coiled-coil of a-helices, also known as a ‘leucine zipper’. The present inventors identified 4 overlapping ‘leucine zipper’ regions conserved in the rat and human sequence, and the high a-helix content of HCaRG makes it a possible DNA-binding protein. The present inventors are currently investigating this possibility. It has been shown that nuclear receptors require the ligand-dependent recruitment of co-activator proteins to effectively stimulate gene transcription (50). The nuclear receptor interaction domain of these factors is highly conserved and contains the consensus sequence LXXLL (where X is any amino acid). This motif is sufficient for ligand-dependent interaction with nuclear receptors (51). The present inventors have identified 1 of these motifs in HCaRG. Nuclear localization of HCaRG protein makes this gene a potential transcription regulator.

Recently, a new transcription factor from the rat kidney (Kid-1) was identified (52-55). It was reported that kid-1 mRNA levels declined after renal injury secondary to ischemia (56). Similarly, decreased HCaRG mRNA levels are seen when epithelial cells are de-differentiated and proliferate (following ischemia and reperfusion). In the model of unilateral ischemic injury, it was shown that contralateral uninephrectomy attenuates apoptotic cell death and stimulates tubular cell regeneration (28-31). The present inventors demonstrate here that HCaRG mRNA levels decreased 3 and 6 h after ischemia in contrast to c-myc expression which is correlated with hyperplastic responses (31). The present inventors also observed that its levels are lower in all fetal organs tested when compared to adult organs, and lower in tumors and the cancerous cell lines tested. It is possible that the gene product may exert a negative effect on growth. This was confirmed by the stable expression of HCaRG in HEK293 cells. The present inventors found that HCaRG overexpression had a profound inhibiting effect on HEK293 cell proliferation. This was shown not only by lower cell number but also by lower DNA synthesis, suggesting that the effect seen was not due to a death promoting effect of HCaRG.

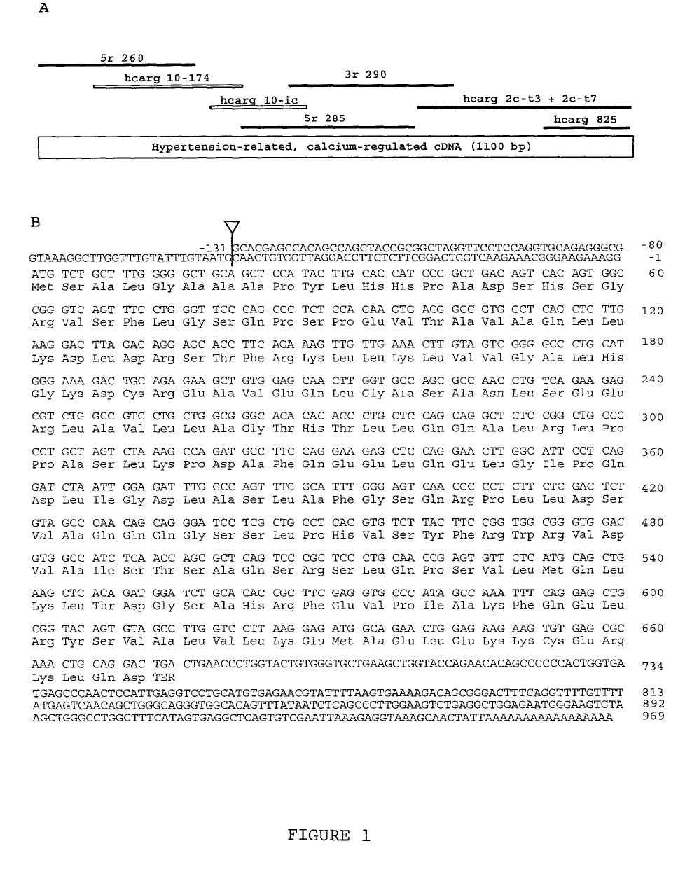

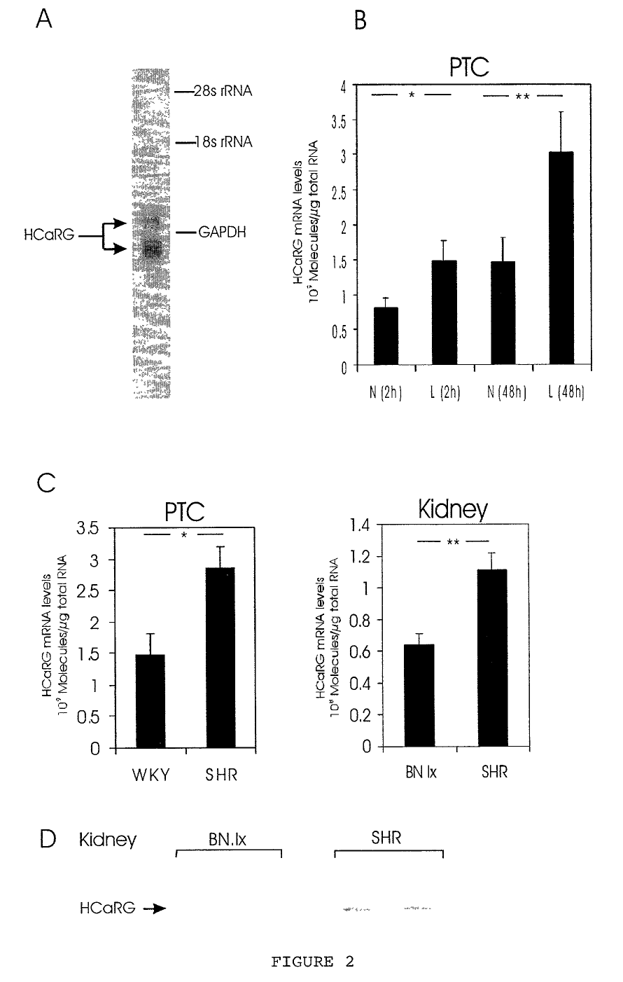

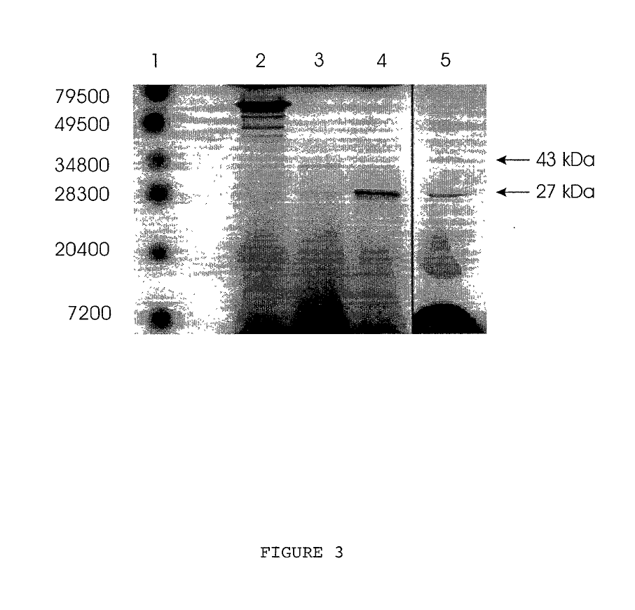

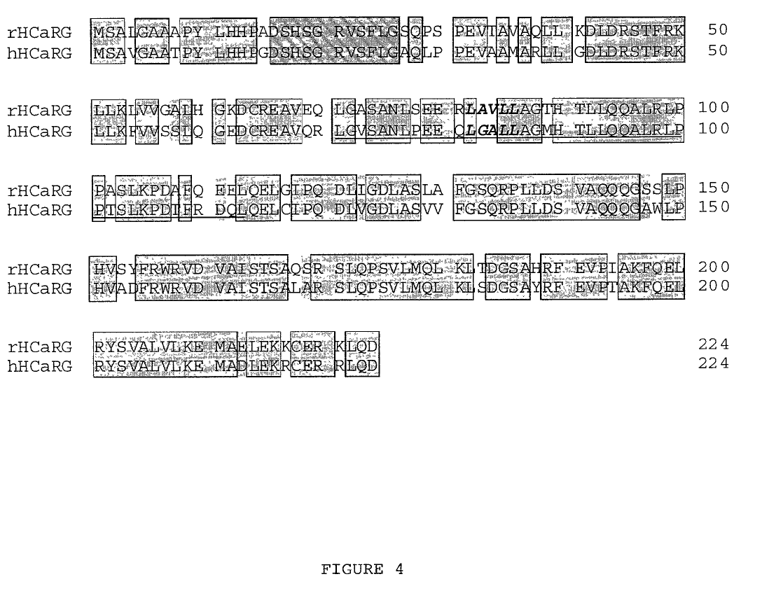

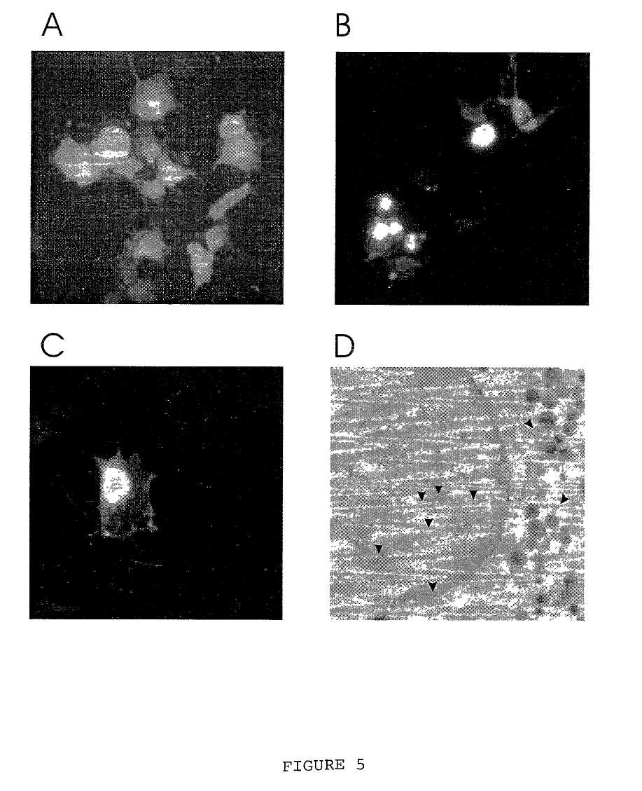

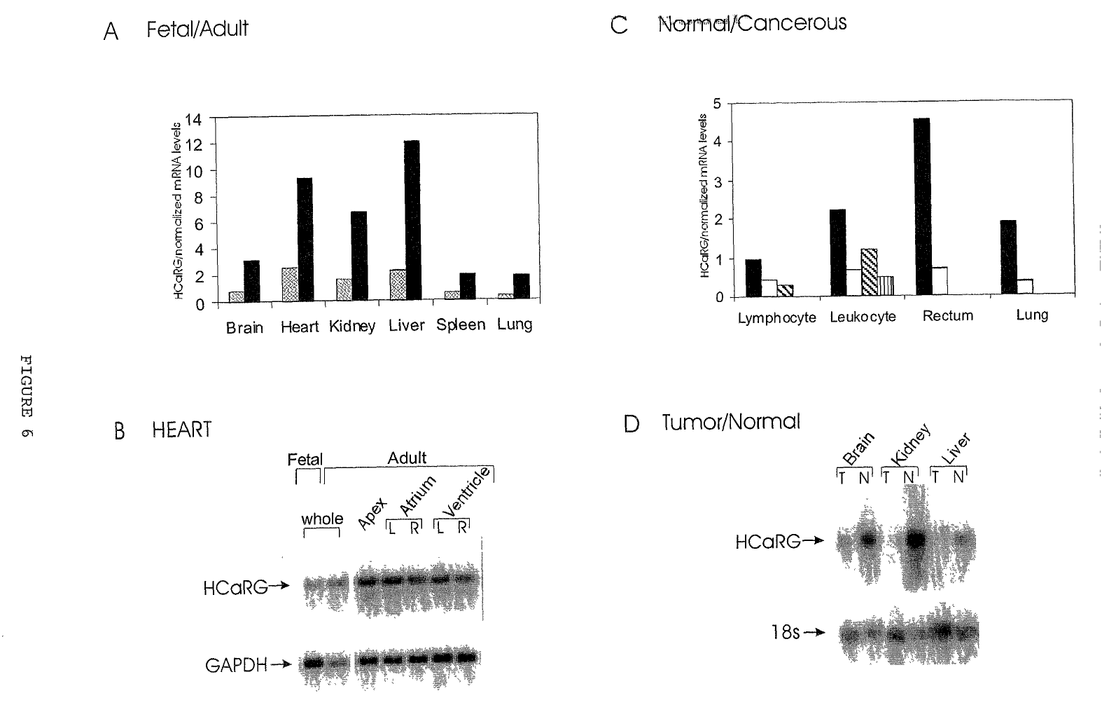

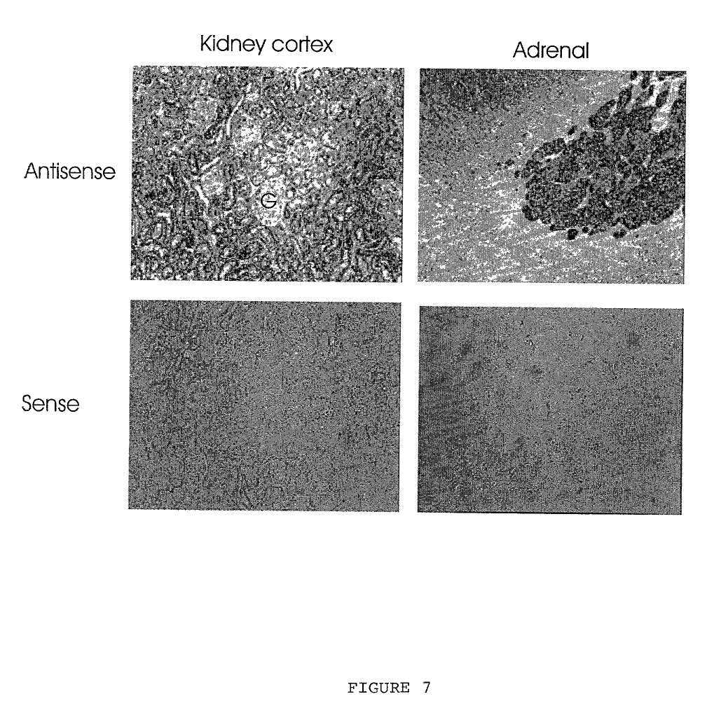

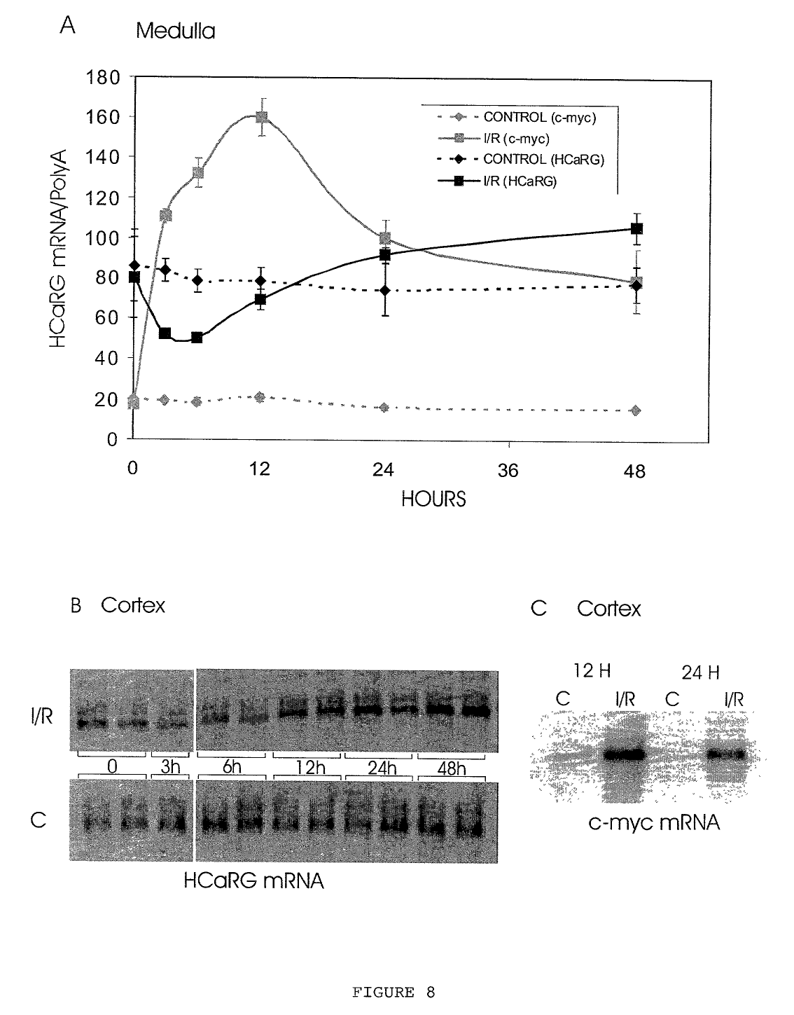

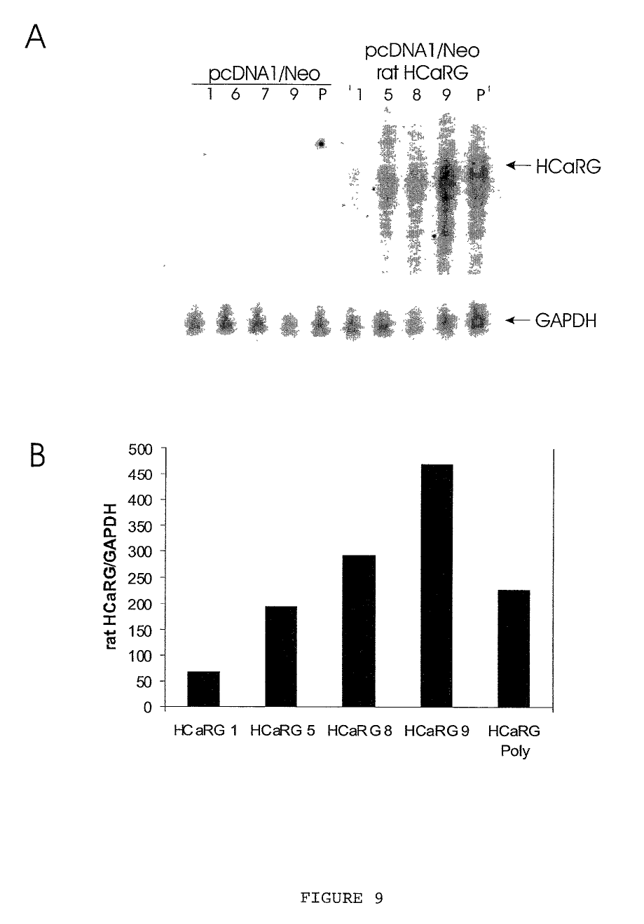

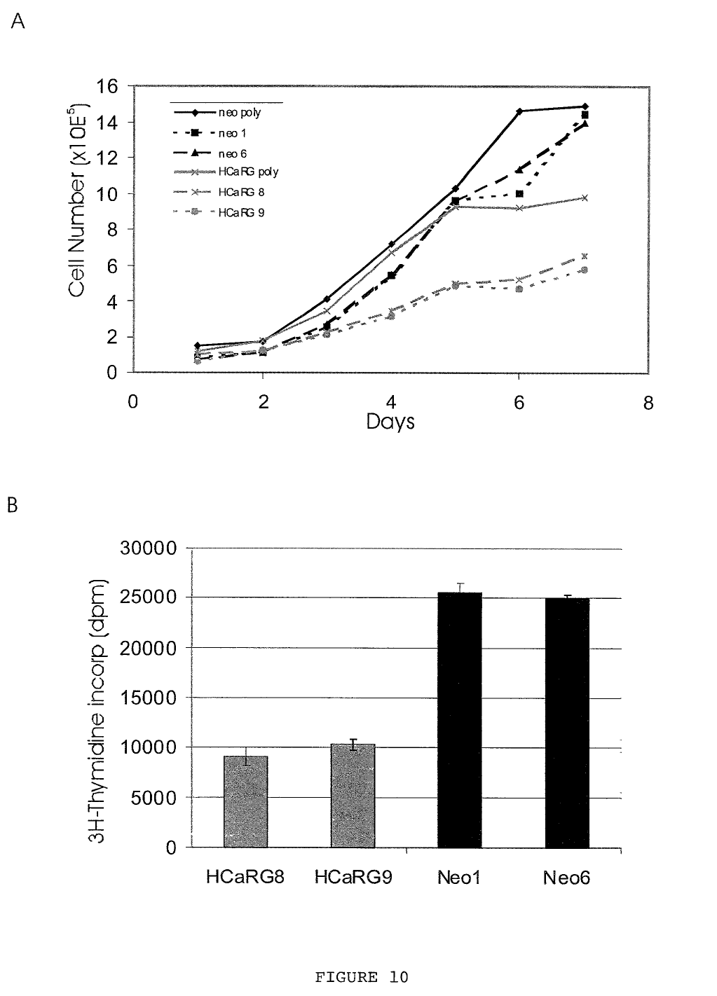

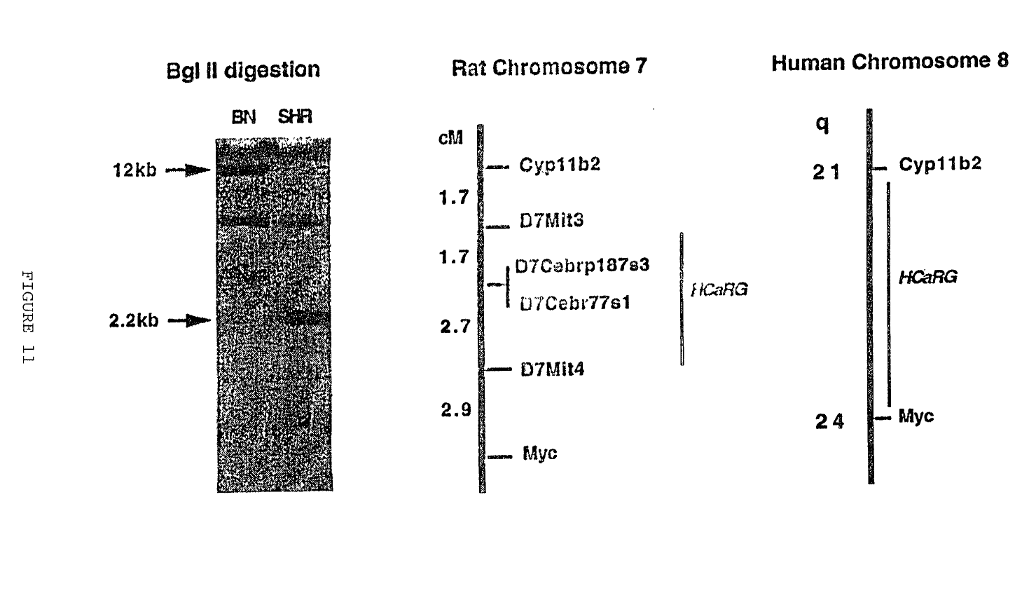

Included within this invention are nucleic acid sequences having 60% or greater homology to all or part of the sequence of the gene for HCaRG of the rat as shown in FIG. 1. Furthermore, this invention includes nucleic acid sequences having 60% or greater homology to all or part of the translated portion of the gene for HCaRG of the rat This would include nucleic acid sequences whose codon usage has been modified to suit a particular host. Sense, antisense and mRNA sequences are encompassed by the term “nucleic acid sequences”.

Also included within this invention are nucleic acid sequences having 60% or greater homology to all or part of the sequence of the gene coding for HCaRG of the human as shown in FIG. 4. Furthermore, this invention includes nucleic acid sequences having 60% or greater homology to all or part of the translated portion of the gene for HCaRG of the human. This would include nucleic acid sequences whose codon usage has been modified to suit a particular host. Again, sense, antisense and mRNA sequences are encompassed by the term “nucleic acid sequences”.

Furthermore, proteins encoded by all or part of the nucleic acid sequences of the gene for HCaRG of the rat and of the human are within this invention. One protein would include the amino acid sequence for the HCaRG protein of the rat as shown in FIG. 4 (top lines). Another protein would include the amino acid sequence for the HCaRG protein of the human as shown in FIG. 4 (bottom lines). Again, proteins having 60% or greater homology to all or part of these proteins are within this invention. It will be appreciated that a protein encoded by the genes of this invention may be modified by substituting amino acids for like amino acids. For example, a basic amino acid may be substituted with a different basic or non-basic amino acid. The substitutions would be chosen so as not alter the properties of the protein encoded by the genes of this invention.

Mimetics of the protein may also be used in the methods and compositions of the invention. The term “mimetic” refers to compounds which have a related three dimensional structure i.e. compounds which have the characteristic structure of the protein encoded by the DNA sequences of this invention. Mimetics may be based on the biologically active portion of the proteins of this invention and may try to mimic the three dimensional structure of that active portion.

There is abnormal calcium transport, concentration and binding in patients with hypertension including calcium leak in cortical tubules. This invention provides additional solutions for patients having hypertension and other diseases caused by abnormal calcium levels.

In addition to hypertension, abnormal modulation of calcium levels can lead to a number of other diseases, disorders or abnormal physical states including hyperthyroidism, osteoporosis, osteopetrosis, heart failure, insulin dependent and independent diabetes, disorders of the central nervous system including stroke, cancer (including breast, thyroid, colon, kidney and leukemia), arteriosclerosis, gastrointestinal diseases, inflammatory bowel disease and asthma. The nucleic acid sequence of this invention could be used (1) for the treatment of diseases related to the modulation in calcium levels, (2) to develop pharmaceutical compositions for the treatment of diseases related to the modulation in calcium levels, or (3) to diagnose diseases related to the modulation in calcium levels. As certain types of cancer are characterized by an increase in intracellular free calcium, the nucleic acid sequence could be used to generate immunological assays (or markers) for these types of cancers and to develop pharmaceutical compositions to treat these types of cancers.

Similarly, all or part of the proteins encoded by the nucleic acid sequences of this invention or antibodies to the proteins could be used to generate immunological assays (or markers) to test for diseases, disorders or abnormal physical states associated with abnormal modulation of calcium levels. The assays could be screening assays to determine whether a product enhances or inhibits calcium levels or whether a product has had its intended effect in enhancing or inhibiting calcium levels.

In the assays of this invention, the complexes may be isolated by conventional methods known to those skilled in the art, such as isolation techniques, for example, chromatography, electrophoresis, gel filtration, fractionation, absorption, polyacrylamide gel electrophoresis, or combinations thereof The complexes or free protein or mimetics may be assayed using known methods. To facilitate the assay, antibody against the protein or mimetic may be labelled or a labelled compound may be used. Delectable markers or labels which would serve to identify the complexes could include fluorescein, HRP and biotin

The invention also relates to pharmaceutical compositions to treat patients having abnormal modulation of calcium levels. The compositions could include (1) nucleic acid sequence for use in gene therapy in which the sense sequence of the HCaRG gene is used in liposomes or a recombinant vehicle, for example, to enhance the gene. (2) nucleic acid sequence for use in gene therapy in which the antisense sequence of the HCaRG gene is used in liposomes or a recombinant vehicle, for example, to suppress the gene, (3) a protein or mimetic which competes with the protein encoded by the nucleic acid sequences of this invention thus suppressing the native protein's effect, (4) a protein encoded by the nucleic acid sequence of this invention to enhance the native protein's effectd. The composition could include an acceptable carrier, auxiliary or excipient.

The pharmaceutical compositions may be used as an agonist or antagonist of the interaction of a protein encoded by HCaRG and a receptor. The compositions can be for oral, topical, rectal, parenteral, local, inhalant or intracerebral use. There may be in solid or semisolid form, for example pills, tablets, creams, gelatin capsules, capsules, suppositories, soft gelatin capsules, gels, membranes, tubelets. The compositions of the invention may also be conjugated to transport molecules to facilitate transport of the molecules.

The pharmaceutical composition can be administered to humans or animals. Dosages to be administered depend on patient needs, on the desired effect and on the chosen route of administration.

The pharmaceutical compositions can be prepared by known methods for the preparation of pharmaceutically acceptable compositions which can be administered to patients, and such that an effective quantity of the active substance is combined in a mixture with a pharmaceutically acceptable vehicle. Suitable vehicles are described, for example in Remington's Pharmaceutical Sciences (Remington's Pharmaceutical Sciences, Mack Publishing Company, Easton, Pa., USA 1985).

On this basis, the pharmaceutical compositions include the active compound or substance in association with one or more pharmaceutically acceptable vehicles or diluents, and contained in buffered solutions with a suitable pH and iso-osmotic with the physiological fluids. The methods of binding the compound to the vehicles or combining them with diluents is well known to those skilled in the art. The composition could include a targeting agent for the transport of the active compound to specified sites within cells, tissues or organs. Compounds could be targeted to cells such as vascular smooth muscle, renal or cardiac cells, for example.

The invention also relates to a composition for use in gene therapy. Liposomes or a recombinant molecule, for example could contain a sense or antisense sequence of the nucleic acid molecule of this invention. In the case of a recombinant molecule, the molecule would contain suitable transcriptional or translational regulatory elements.

Suitable regulatory elements may be derived from a variety of sources, and they may be readily selected by one or ordinary skill in the art. If one were to upregulate the expression of the gene, one would insert the sense sequence and the appropriate promoter into the vehicle. If one were to down regulate the expression of the gene, one would insert the antisense sequence and the appropriate promoter into the vehicle. These techniques are known to those skilled in the art

Examples of regulatory elements include: a transcriptional promoter and enhancer or RNA polymerase Winding sequence, a ribosomal binding sequence, including a translation initiation signal. Additionally, depending on the vector employed, other genetic elements, such as selectable markers, may be incorporated into the recombinant molecule, The recombinant molecule may be introduced into cells of a patient using in vitro delivery vehicles such as retroviral vectors, adenoviral vectors, DNA Virus vectors and liposomes. They may also be introduced into such cells in vivo using physical techniques such as microinjection and electroporation or chemical methods such as coprecipitation and incorporation of DNA into liposomes. The compositions may also De delivered in the form of an aerosol or by lavage.

The present invention also provides for methods in which a patient suffering from a condition requiring modulation of calcium levels is treated with an effective amount of a composition.

List of abbreviations

ANP atrial natriuretic peptide

BN.lx Brown-Norway rats

DMEM Dulbecco's modified Eagle's medium

FBS Fetal bovine serum

FCS Fetal calf serum

GFP Green fluorescent protein

GST Glutathione S-transferase

HCaRG Hypertension-Related, Calcium-Regulated Gene

IP3 Inositol 1,4,5 trisphosphate

MTE Multiple tissue expression

nCARE Negative calcium-responsive element

PAGE Polyacrylamide gel electrophoresis

PBS Phosphate-buffered saline

PCR Polymerase chain reaction

PHF Parathyroid hypertensive factor

PTC Parathyroid cells

PTH Parathyroid hormone

RACE Rapid amplification of cDNA ends

RT Reverse transcription

SDS Sodium dodecyl sulfate

SHR Spontaneously hypertensive rat

SSC Standard sodium citrate

VSMC Vascular smooth muscle cells

WKY Wistar-Kyoto rats

ZFP7 Zinc finger protein 7.5

### EXAMPLE 1

### Isolation of a Novel cDNA Whose Expression is Negatively Regulated by Extracellular Calcium in the SHR Parathyroid Gland

Using sense candidate primers (from a putative amino acid sequence of PHF (24)) and a hybrid oligo dT primer, 3′-RACE experiments, performed on total RNA extracted from SHR PTC cultured in low-calcium medium. generated 1 major 700-bp fragment that was digested and cloned in the BamH I site of pSP72. As a BamH I site was present in the 700bp fragment, a recombinant plasmid containing a 300-bp insert was isolated and sequenced. This fragment was used to screen the PTC library and to generate new oligonucleotide primers to extend the cDNA towards the 5′- and 3′-ends by RACE. From 7 overlapping DNA fragments isolated in the above experiments and from SHR PTC cDNA library screening, a 1100-bp EDNA was reconstituted (FIG. 1A). The rat 1100-bp reconstituted cDNA sequence contained an open reading frame of 224 codons preceded by 2 in-frame stop codons and followed by the most frequent variant of the poly A tail (FIG. 1B) A 342-bp intron was localized at position -52 from the translation initiation site.

Poly A RNA was isolated as described and analyzed by Northern hybridization with the 32 P labeled 300-bp fragment (FIG. 2A). Two bands were detected with this probe, at approximate lengths of 1.2 and 1.4 kb. These results suggest either the existence of 2 genes or differential splicing. Furthermore, they indicate that the reconstituted 1100-bp cDNA is almost full length cDNA, estimated at 1.2 kb by the major band in the northern hydridization experiments.

Regulation of the expression of this novel gene was investigated by competitive RT-PCR assay in PTC from WKY and SHR. Cells between 5 and 12 passages were tested in these studies. In WKY PTC, lowering of ambient calcium from 2.0 mM to 0.3 mM induced a rapid 2-fold increase in the mRNA levels of this novel gene at 2 h, which lasted up to 48 h (FIG. 2B). This calcium regulation was detected in WKY PTC up to about 12 passages but disappeared in long term cultures. Lowering of calcium concentrations in the cell medium also increased the mRNA levels of this novel gene in SHR PTC but to a lesser extent than in WKY cells (data not shown) The present inventors then compared its mRNA levels between 2 normotensive At strains (Brown Norway, BN.lx, or WKY) and hypertensive animals (SHR). The present inventors observed that the mRNA levels of this novel gene were significantly higher in PTC derived from SHR (FIG. 2C left panel) compared to normotensive WKY rats at normal calcium. Similarly, when the present inventors extracted RNA (FIG. 2C right panel) or proteins (FIG. 2D) directly from the kidneys, the present inventors found significantly higher levels of this novel gene in hypertensive rats. These results clearly show that this novel gene is negatively regulated by extracellular calcium concentrations and that its levels are significantly higher in genetically hypertensive rats compared to 2 normotensive strains The present inventors, therefore, named this gene Hypertension-related, Calcium-Regulated Gene (HCaRG).

### EXAMPLE 2

### Sequence and Structure of HCaRG cDNA

The deduced protein contained 224 amino acids with a calculated molecular weight of 22456 Da. The estimated pl of the protein was 6.0. It comprised no known membrane-spanning motif but had an estimated 67% a-helix content. The absence of a putative signal peptide sequence suggested an intracellular protein. There were 2 cysteines in the sequence, indicating possible intra- or inter-molecular disulfide bridges (Cys 62-cys 218). The protein had several putative phosphorylation sites for C- and A-kinases and 1 potential Asn-glycosylation site (Asn 76). To confirm that HCaRG mRNA encodes a peptide of expected size, the HCaRG cDNA inserted into pSP72 was incubated in vitro in a coupled transcription/translation labeling system. It was transcribed by T7 RNA polymerase, and translated in rabbit reticulocyte lysate As shown in FIG. 3 (lane 4), HCaRG mRNA directed the synthesis of a peptide with a molecular mass of 27 kDa which closely corresponded to the molecular weight calculated from the amino acid sequence. PAGE analysis of the reaction product in the absence of the reducing agent β-mercaptoethanol showed bands of 27 and 43 kDa (FIG. 3, lane 5) These results suggest possible intramolecular or intermolecular disulfide badges and the formation of homodimers or heterodimers with other protein(s) present in the lysate.

### EXAMPLE 3

### Cloning of Human HCaRG

The present inventors then used a 439-bp cDNA fragment of rat HCaRG (+1 to +440 in FIG. 1) to screen a human VSMC cDNA library. The present inventors identified several positive clones that were purified, subcloned in pBluescript vector and sequenced. The present inventors obtained a 1355-bp sequence containing full length human cDNA, while all other clones contained only partial sequences. A recent sequence search in Genbank revealed a region with complete DNA sequence homology within 3 cosmids containing the zinc finger protein 7 (ZFP7) gene (accession numbers AF124523, AF146367 and AF118808). Although the nucleotide sequence of human HCaRG could be found in these cosmids, the present inventors are the first to assign an expressed gene sequence to this DNA region.

Sequence comparison between human HCaRG and rat HCaRG showed 80% identity at the nucleotide level (data not presented) and, similarly, 80% homology at the amino acid level (FIG. 4). Analysis of protein structure with the PROSEARCH database revealed 4 overlapping putative leucine zipper consensus motifs (FIG. 4 underlined). Further analysis revealed homology to the EF-hand calcium-binding motif (8 out of the 10 most conserved amino acids) (FIG. 4 dashed box) We also identified a nuclear receptor-binding motif (FIG. 4 bold and italics). All these motifs were conserved in the rat and human amino acid sequence.

### EXAMPLE 4

### Subcellular Localization of HCaRG

The present inventors expressed GFP-HCaRG in COS-7 cells. Fluorescence study showed that GFP-HCaRG localized in the nucleus while coplasmic fluorescence was very faint (FIG. 5B). GFP, on the other hand, had a very diffuse localization (FIG. 5A). This result was confirmed by immunofluorescence using antibodies specific to HCaRG (FIG. 5C) and by electron microscopy (FIG. 5D). Electron microscopy was also performed on different tissues. In all tissues studied, HCaRG was round in the nucleus With some labeling in protein synthesis sites.

### EXAMPLE 5

### HCaRG Expression in Various Human Tissues

A human MTE TM array was hybridized with human 32 P-labelled HCaRG cDNA as a probe. The array contained 76 polyA RNAs from various adult tissues, cell lines, fetal tissues and cancerous cell lines. These arrays were normalised to 8 different housekeeping genes. Analysis of the array showed that HCaRG was expressed preponderantly in the heart, stomach, jejunum, kidney, liver and adrenal glands. Comparison of HCaRG expression in fetal to adult organs revealed that HCaRG mRNA was less expressed in all fetal tissues compared (FIG. 6A), particularly in the heart, kidney and river. Northern blots confirmed the lower abundance of HCaRG in the fetal heart compared to all regions of the adult heart (FIG. 6A). The present inventors also compared HCaRG mRNA levels in various cancerous cell lines to normal tissues (FIG. 6C). HCaRG mRNA levels were decreased in all cancerous cell lines studied. They were also much lower in a glioblastoma, a partly differentiated renal cell carcinoma and a moderately differentiated hepatocellular tumor compared to the same amount of normal RNA of adjacent tissues excised from the same operational site (FIG. 6D).

### EXAMPLE 6

### In situ Hybridization of HCaRG mRNA in the Kidney and Adrenal

HCaRG expression was determined in SHR tissues by in situ hybridization. The labeled antisense riboprobe hybridized to the medulla and zona fasciculata of the adrenal cortex (FIG. 7). In the kidney, labeling was almost exclusively located in the cortex and concentrated in the tubular component, contrasting with virtual absence of the signal in glomeruli (FIG. 7). In these organs, the signal was clearly greater in hypertensive rats compared to their normotensive controls (Lewanczuk et al; unpublished data). The sense probe was used as a negative control and appropriately revealed a low signal under the present inventors' hybridization conditions, demonstrating specificity of the reaction (FIG. 7 lower panels).

### EXAMPLE 7

### HCaRG mRNA Levels After Ischemia-reperfusion

The process of kidney injury and repair recapitulates many aspect of development. It involves de-differentiation and regeneration of epithelial cells, followed by differentiation (25-27). Since the present inventors observed that HCaRG mRNA levels are lower in fetal than in adult organs, the present inventors evaluated HCaRG expression after unilateral renal ischemia in uninephrectomized rats (19) as contralateral nephrectomy has seen shown to stimulate cell regeneration (28-31). The present inventors noted that HCaRG mRNA declined rapidly to its lowest levels at 3 h and 6 h of reperfusion (FIG. 8A). These values then increased steadily to higher than baseline at 48 h of reperfusion. This was observed in both the kidney medulla (FIG. 8A) and cortex (FIG. 81). In contrast to the decline in HCaRG mRNA levels, the proto-oncogene c-myc expression, which is correlated with hyperplastic response in mammalian cells, was rapidly increased following renal ischemia and reperfusion (31). c-myc mRNA levels were low in control kidneys and increased dramatically in the post-ischemic Kidney at 3 h of reperfusion, at a time when HCaRG mRNA levels were already reduced (FIG. 8A and 8C)

### EXAMPLE 8

### Overexpression of HCaRG Inhibits Cell Proliferation

HEK293 cells were stably transfected with either plasmid alone or with plasmid containing rat HCaRG. After transfection, several clones were examined for the determination of rat HCaRG mRNA levels. Four clones (HCaRG clones 1, 5, 8 and 9) expressed variable amounts of rat HCaRG mRNA, as detected by northern blots, while no HCaRG mRNA levels were found in clones transfected with the plasmid alone (FIG. 9). Clones expressing the highest levels of HCaRG (clones 8 and 9) were selected for cell proliferation studies. For these studies, cells that were transfected with the vector alone or polyclonal HCaRG-transfected cells served as controls. The proliferation rates of the HCaRG-transfected cell lines and vector control cells were examined under normal growth conditions (10% FGS and G-418) by counting cell numbers every day for a period of 8 days after plating. Cell lines transfected with the vector alone (Neo clones 1 and 6) showed a similar growth rate as non-transfected cells (not presented). Clones 8 and 9 expressing high levels of rat HCaRG revealed a much lower proliferation rate than vector control cells while polyclonal cells expressing intermediate values of HCaRG fell in between (FIG. 10A) Consistent with a lower proliferation rate, stable HCaRG transfection clones 8 and 9 showed much lower 3 H-thymidine incorporation than clones transfected with the empty vector (FIG. 10B).

### EXAMPLE 9

### Enhanced Sensibility to Cell Death by Apoptosis and Necrosis

In order to investigate the cellular function of HCaRG, we have studied the effects of ectopic overexpression of HCaRG protein in HEK 293 cells. Stably transfected cell lines which expressed either plasmid alone (pcDNAI/Neo) or plasmid containing rat HCaRG (pcDNA1/Neo-HCaRG) were used in these studies. The level of [3H]-Thymidine incorporation was significantly lower in HCaRG transfected clones compared to the vector control cell lines. Cell cycle analysis revealed a G₂M phase accumulation of HCaRG cells suggesting a cell cycle-dependent mechanism of growth suppression, which was associated with upregulation of the cyclin dependent kinase (cdk)-inhibitor p21Cip1/WAF-1, both at the mRNA and protein level. The reduced cell proliferation was associated with some enhanced sensitivity to cell death by apoptosis and necrosis which was apparently secondary to cell cycle-dependent G₂M phase accumulation. HCaRG transfected cells had a larger size and a greater total protein content per cell, consistent with cellular hypertrophy. Previous studies, including those using immunohistochemical techniques, have demonstrated Atrial natriuretic peptide (ANP) is present in the tubules of kidneys of several species including rat and human in vivo (68-70). Furthermore, the developmental pattern of ANP immunoreactivity in the rat was studied and found to coincide with the differentiation and maturation of the tubular epithelium (68). Additional studies have provided evidence that an ANP-like peptide is produced and secreted by primary cultures of neonatal and adult rat kidney cells (71,72). The human embryonic kidney cell line (HEK 293) is derived from renal cortical cells and exhibits several phenotypic characteristics of renal distal tubular cells, including a basal synthesis and release of an ANP-like immunoreactivity (or Urodilatin) (73). We assessed the direct functional effects of the novel gene HCaRG, on cellular proliferation, cell cycle regulation and cell phenotype in vitro Since the HEK 293 cell line is considered to be most representative of natriuretic peptide (NP)-secreting human distal cortical tubular cells, we have stably transfected these calls with HCaRG in order to assess the direct effect of ectopic HCaRG expression on several aspects of renal epithelial cell function in vitro. Overexpression of the HCaRG gene caused a 6-8 fold increase in the rate of ANP release from HEK 293 cells. Light and electron microscopy revealed a lower incidence of mitotic figures as well as the development of more differentiated junctions in HCaRG transfected cells only In conclusion, HCaRG gene transfer to HFK 293 cells in vitro caused a change in cell phenotype which was manifest as: a reduction in cell growth, increased cell doubling time; cell cycle G₂M phase accumulation; increased cell size and total protein content per cell and increased synthesis and secretion of an ANP-like immunoreactivity. Taken together, all of these findings are consistent with the hypothesis that HCaRG can suppress cell proliferation in a cell cycle-dependent manner, and induce features characteristic of differentiation in vitro, apparently by affecting cell cycle progression which is associated with up-regulation of p21^{Cip1AA/AF-1}.

### EXAMPLE 10

### HCaRG Expression in Mammalian Cells

Because bacteria are unable to post-translationally modify proteins as mammalian cells, a bacterial protein may be inactive. We express the HCaRG protein in mammalian cells to circumvent this problem. Gene transfer techniques to COS7 cells are used routinely in the lab. The expression vector is pcDNAneol (Invitrogen) available in the lab. The cloned HCaRG is inserted as a Hind III Bgl II fragment in Hint III-BamHI sites in the vector to place the gene under the CMV promoter. A plasmidic neo gene enables the selection of stable transformants.

High expression is selected by Northern blots and protein is purified when antibodies are available. Various biological activities however, are tested immediately on cells expressing HCaRG. The initial candidate activities are calcium channel function calmodulin-phosphodiesterase activator activity, cell proliferation, cell death and apoptosis.

### EXAMPLE 11

### Gene Therapy—The Intracellular Function of a Protein Can be Also Studied by Inhibition of its Expression by Antisense Molecules

Recently, antisense oligonucleotides have been used extensively to inhibit expression of specific genes (65). Although, the exact mechanism of this inhibition is not known, evidence suggest that Rnase H-like activity degrades RNA oligonucleotide duplexes (61). While modified oligonucleotides such as methylphosphonates diffuse freely across the cell membrane, unmodified and modified oligonucleotides have been shown to be actively transported into living cells by binding to membrane receptors (63, 66). It is therefore possible to inhibit the expression of specific genes and their gene products by adding specific antisense molecules to the culture medium. We explore the capacity of the oligonucleotide antisense spanning the translation initiation site of HCaRG to inhibit PHF as well as CPA synthesis Parathyroid cells or other cells expressing HCaRG, PHF or CPA are treated with antisense oligonucleotides. Cells are incubated in medium containing up to 100 μM antisense oligonucleotide. Lipofection helps to increase the percentage of uptake of oligonucleotides in certain cells. Fresh antisense molecules are added every 24 hrs. After 24 to 48 hrs cell culture medium is tested for the presence of PHF activity and intracellular CPA activity is assessed. Non-sense and sense oligonucleotides are used as control for determination of specificity of the effect. Other parameters such as cyclic nucleotides and intracellular calcium levels are also measured since they may constitute an additional step to define the mechanism of action of HCaRG.

### EXAMPLE 12

### TPA, a Protein Kinase C Agonist Increases mRNA Levels

We have initiated studies on the regulation of HCaRG. Hormonal signal transduction pathways are stimulated by different agonists, in cultural parathyroid cells incubated in low or normal calcium medium. Our initial studies showed that TPA, a protein kinase C agonist, (Protein kinase C is the main target on intracellular calcium and is involved in the phosphorylation regulation of many target proteins including, ionic channel, contractile proteins and hormonal receptors) increases the mRNA levels of HCaRG when cells are incubated in normal calcium medium. These data suggest that Protein Kinase C could mediate, inside of the cell, these effects of extracellular calcium. Interestingly, the calcium sensor is linked to the protein kinase C pathway. Other hormonal systems are tested for their effects on HCaRG expression. These include glucocorticoids, catecholamines, Vitamin D, parathormone, growth factors, cytokines. These tests define the mechanisms controlling HCaRG synthesis and delineate their anomalies in disease states.

### EXAMPLE 13

### Chromosomal Localization

With the obtention of the cRNA coding for the HCaRG from human and rat and the putative full length open reading frame, our research includes genomic structure, search of genetic control elements. Our research relates to the pathophysiological regulation of its expression and to in vitro expression of a functional protein.

Southern Plot analysis was performed on 10 μg genomic DNA of SHR and BN.lx rats with the following restriction enzymes BamH 1, Bgl II, EcoR 1, Hind III, Kpn I and Pst 1. The probe consisted of the ³²P-labeled fragment of 860bp of HCaRG shown in FIG. 1. A clear RFLP genotyping for the B BN.lx allele (12 kb) or S (SHR) allele (2.2 kb) was then detected with the Bgl II restriction enzyme (FIG. 11) in the 33 recombinant inbred strains. The strain distribution pattern of this RFLP was then analyzed by Pearson's correlation for segregation with 500 markers localizes in the rat genetic map using the Map Manager program of Manly (version 2.6.5). The address of RATMAP is “http://www ratmap.gen.gu.se”.

### EXAMPLE 14

### Pathophysiological Regulation of Expression of HCaRG

Northern blot and in situ hybridization experiments have shown that rat tissues which demonstrates a significant expression of HCaRG are the parathyroid gland, the medulla and inner cortical section of the adrenal gland, the cortical tubular segments of the kidney and the brain cortex and medulla. In most organs, the expression was higher in SHR than in normotensive rat. The effect of dietary sodium and calcium is tested on HCaRG expression in these organs in salt sensitive and salt-resistant hypertensive rat strains with a protocol previously described in Chang et al, (60) and Tremblay et al. (67). These earlier reports have shown an increased in CPA activity by high sodium intake and normalization by high dietary calcium suggesting that this factor could be a biological marker of salt sensitivity in the population. We have recently detected the expression of HCaRG in human lymphocytes. This is a readily available source of human RNA and we have developed a semi-quantitative RT-PCR assay to quantify the mRNA levels of HCaRG in humans. Human samples are obtained from controls and patients with abnormal calcium metabolism such as patients with cardiovascular diseases, osteoporosis, atherosclerosis and cancer. In addition, biopsies of cancer tissues are obtained. We have already detected the mRNA of HCaRG in colon cancer as well as in breast cancer. These studies use HCaRG as a biological marker of abnormal calcium metabolism in humans.

### EXAMPLE 15

### HCaRG Expression in Bacteria and Antibody Preparation

Rat and human HCaRG are inserted into bacterial expression vectors in order to produce large amounts of HCaRG protein. For HCaRG, we use the pMAL-c2 (New England Biolabs) to generate a fusion protein of HCaRG following the maltose-binding protein. A blunt HCaRG cDNA obtained by PCR and starting at the initiator methionine is inserted in the Xmnl site of pMAL-c2. This strategy places the HCaRG product next to the Factor Xa cleavage site of the fusion protein.

Because protein expression in E. coli varies according to the vector used and the nature of the protein expressed, we prepare other fusion proteins The pGF-X-5×plasmid (Pharmacia) allows for the introduction of genes to produce glutathion S-transferase fusion proteins. The vector exists in three frames and has extensive restriction insertion sites for easy insertion of foreign gene For example, the cloned rat HCaRG is inserted in the EcoRI-XhoI sites to produce the fusion protein with HCaRG product localized after a Factor Xa cleavage site. In both systems, the fusion proteins enable the rapid purification of the expressed protein through affinity chromatography. Crude bacterial extracts containing cytoplasmic proteins are analysed. According to the amount of protein synthesized, purfication steps are determined or crude extract is used directly. To generate antibodies by injection into rabbits, urea extracted aggregates, SDS-page purified bands or protein exacts are used (64).

### EXPERIMENTAL PROCEDURES

### Cell Cultures

Parathyroid cells (PTC) were isolated from SHR and Wistar-Kyoto (WKY) rats. Primary cultures were passaged in Dulbecca's modified Eagle's medium (DMEM) with 10% fetal calf serum (FCS), as described previously (0). They were then maintained in Ham F12 medium containing a low (0.3 mM) or normal (2.0 mM) total calcium concentration for 2 or 48 h. COS-7 or HEK293 cells were cultured in DMEM containing 10% fetal calf serum. All cell types were maintained in 5% CO2 at 37° C.

### Ischemia-reperfusion

SHR were anesthetized with light flurane, and the tight kidney was removed through a mid abdominal incision. The left kidney was subjected to warm transient ischemia by occlusion of the left renal artery and vein with a micro-clip, as described previously (19). The skin incision was temporarily closed. After 60 min of occlusion, the clip was removed, and the wound was closed with a 2-0 suture. The rats had access to water immediately after surgery.

### SHR Parathyroid cDNA Library

Parathyroid glands were removed from 100 12-week-old SHR and frozen immediately in liquid nitrogen The glands were added to a guanidinium thiocyanate solution and homogenized in this solution. Poly A RNA was obtained by phenol-chloroform extraction, ethanol precipitation and isolated on an oligo(dT) column. Poly A RNA was stored in ethanol at −80° C. until used. The cDNA library was constructed with Poly A RNA as template and the ZAP-cDNA synthesis kit Stratagene, La Jolla, U.S.A.). A summary of the protocol is as follows: mRNA was reverse-transcribed from an XhoI-linker oligo(dT) primer using Moloney-Murine leukemia virus reverse transcriptase. Second strand synthesis was then produced with DNA polymerase I in the presence of RNaseH. The cDNA was then extracted using phenol-chloroform, precipitated with sodium acetate, washed with 80% ethanol and resuspended in sterile water. cDNA termini were blunted by incubation with the Klenow fragment of DNA polymerase I and dNTPs. cDNA was again precipitated and washed. EcoRI adaptors were added using T4 ligase, and the ends phosphorylated with T4 polynucleotide kinase. This mixture was then digested with Xho I to release adaptors and residual linker-primer from the 3′ end of the cDNA. The resulting mixture was separated on a Sephacryl S-400 column. Eluted cDNA was precipitated with 100% cold ethanol and resuspended in sterile water. cDNAs were ligated into the Uni-ZAP XR vector using T4 DNA ligase, thus forming the cDNA library, and packaged into Gigapack II Gold packaging extract. The packaged products were plated onto XL1-Blue MRF' cells and recombinant numbers determined. The library was then amplified by mixing the packaging mixture with host bacteria (XL1-Blue MRF' cells). The library was stored at −80° C. until screened. To screen the cDNA library, phages were plated onto bacterial host plates (XL1-Blue MRF') and incubated overnight. After chilling at 4° C. for 2 h, a nitrocellulose filter was overlaid for 2 min. The filter was then denatured in 1.6M NaCl/0.5M NaOH, neutralized in 1.5M NaCl with 0.5 Tris-CI (pH 8.0). The filter was then rinsed and the DNA crosslinked to it with UV light. Hybridization was performed with digoxigenin-dUTP labeled probes (Roche Molecular Biochemicals, Laval, Canada) derived from 3′- and 5′-RACE (rapid amplificaton of cDNA ends), products described below.

### RNA and cDNA Preparation

Total RNAs were prepared from rat cells and organs according to the standard guanidinium thiocyanate-phenol-chloroform method (20) and kept at −70° C. until used. mRNA was extracted from total RNA with the PolyATtract system (Promega, Nepean, Canada). mDNAs, unless stated, were synthesized with random hexamers for first strand synthesis and reverse-transcribed. Radiolabeled DNA probes were prepared by the random priming technique or polymerase chain reaction (PCR) amplification with 32 P-dCTP.

### 3′ or 5′-RACE

Four mixtures of degenerate oligonucleotide primers were initially designed according to the putative amino acid sequence of PHF with the following degenerate sequence: 5′ TA(T/C) TCI GTI TCI CA(T/C) TT(T/C) (A/C) G 3′. From initial RACE experiments (described below), 1 unique sequence primer TAC TCC GTG TCC CAC TTC CG was selected for its ability to generate reverse transcription (RT)-PCR DNA fragments from PTC total RNA and used subsequently as candidate primer for 3′-RACE. In brief, for 3′-RACE, total RNA from PTC was reverse-transcribed with a hybrid primer consisting of oligo(dT) (17 mer) extended by a unique 17-base oligonucleotide (adaptor). PCR amplification was subsequently performed with the adapter, which bound to cDNA at its 3′-ends, and the candidate primer mentioned above (21). For 5′-RACE, RT was undertaken with an internal primer derived from the sequence of the cDNA fragment generated by 3′-RACE. A dA homopolymer tail was then appended to the first strand reaction products using terminal deoxynucleotidyl transferase. Finally, PCR amplification was accomplished with the hybrid primer described previously and a second internal primer upstream to the first one (21).

### Subcloning

The DNA fragments generated from the RACE experiments were separates by electrophoresis, isolated from agarose gel and extracted by the phenol-chloroform method (20). pSP72 plasmid (Promega) was digested at the Sma I site and ligated to blunt DNA fragments with T4 DNA ligase. Transformed DH5a E. coli bacteria were grown and recombinant bacteria were selected by PCR. Similarly, HCaRG was subcloned in pcDNA1/Neo (Invitrogen, Carlsbad, U.S.A.).

To determine the subcellular localization of HCaRG protein in mammalian cells, the coding region of HCaRG was fused to green fluorescent protein (GFP) cDNA and was transfected in the cells. Briefly, the entire coding region of HCaRG was amplified by PCR with the primers ATG TCT GCT TTG GGG GCT GCA GCT CCA TAC TTG CAC CAT CCC and TAA TAC GAC TCA CTA TAG GGA GAC, gel purified, and fused in-frame to GFP in pEGFP-C1 (Clontech, Palo Alto, U.S.A.) through a blunt Hind III site. pEGFP-HCaRG was then sequenced. Similarly, the coding sequence of HCaRG was fused in frame to glutathione S-transferase (GST) in pGEX-3X (Amersham Pharmacia Biotech, Baie d'Urfée, Canada) through a Sma I site and a blunt EcoR I site.

### Sequencing

Double-stranded sequencing of cloned cDNA inserts was performed with Sequenase Version 2.0 (United States Biochemical, Cleveland, U.S.A.). 5 μg of recombinant plasmid template were denatured, annealed with T7 or SP6 primers, and labeled with 35 S-dATP by extension, using the chain termination method of Sanger according to the manufacturers protocol.

### Cloning of Human HCaRG

A 439-bp cDNA fragment of rat ?1HCaRG was 32 P-labeled and served as a probe for screening a human VSMC cDNA library. DNA from positive phages was purified and the fragments were cloned in pBluescript. All fragments were sequenced. We obtained a 1355-bp fragment containing the coding region of HCaRG.

### Northern Blot Hybridization, Dot Blot Hydridization and Competitive RT-PCR

2 μg of poly A RNA from PTC or 10 μg of total RNA from kidneys were denatured at 68° C. and separated on denaturing formamide 1% agarose gel. The gel was transferred onto nitrocellulose by vacuum transfer with 20×SSC. The membrane was exposed to UV light to fix RNA, and pre-hybridized in a solution containing SSPE, SDS, Denhardt's and dextran sulfate for at least 4 hours. Hybridization was performed overnight in the same buffer containing ³²P labeled probes generated from cDNA clone(s) by PCR or random labeling method. 1 μq of total RNA was used in dot blot experiments. A human multiple tissue expression (MTE TM ) array (Clontech) and human fetal and tumor panel Northern Territory TM RNA blots (Invitrogen, Carlsbad, U.S.A.) were hybridized with 32 P-labeled human HCaRG cDNA according to the manufacturers specifications. For quantitative determinations of HCaRG mRNA, total RNA was extracted from PTC and reverse-transcribed. A HCaRG competitor was constructed using the PCR Mimic Construction Kit (Clontech) With the following composite primers: GCA CGA CCC ACA GCC AGC TAC CCC AGC CAC CCA TTT GTA CC (sense) and TGT GAC TGT CAG CGG GAT GGA GTC CGA GAT GTA GAG GGC (antisense). The 344-bp DNA obtained was cloned into pSP72 and transcribed with SP6 RNA polymerase. The resulting RNA was quantified by photometry and subsequently used in competitive RT-PCR. The competitive reaction contained 1 or 2 μg total RNA with increasing amounts of competitor cRNA along with 32 P-labeled nucleotide. Two primers TGT GAC TGT CAG CGG GAT GG and GCA CGA GCC ACA GCC AGC TACC flanking the HCaRG intron were employed to amplify a 186-bp cDNA fragment. PCR was performed: 15 sec at 95° C., 20 sec at 68° C., 30 sec at 72° C., for 30 cycles, followed by a 5-min elongation step at 72° C. 10 μl of the PCR were loaded on 1.8% agarose gel, then dried and exposed in a PhosphorImager cassette for quantification.

### In situ mRNA Hybridization

Tissues from SHR and WKY rats were rinsed in phosphate buffer, fixed in 4% paraformaldehyde and embedded in paraffin. 3 to 5 μm sections were cut and mounted on microscope slides pretreated with aminopropylthiethoxysilane. The slides were first dried at 37° C., then at 60° C. for 10 min prior to use. The probe applied was a unique 3′-RACE 300-bp fragment (3r 290 in FIG. 2A) which had been subcloned into the BamH I site of a pSP72 vector. Briefly the DNA was purified and linearized with HindIII and EcoR1 digestion followed by phenol-chloroform extraction. After gel confirmation, the DNA was transcribed using T7 or SP6 polymerases to create sense and antisense riboprobes which were labeled with dioxigenin-UTP using a tailing reaction. They were validated by dot blot hybridization with template DNA Prehybridization of slides was undertaken after de-waxing in xlene, followed by progressive ethanol-water hydration (95% to 50%). The slides were rinsed in phosphate-buffered saline (PBS) and incubated with proteinase K (20 μg/ml) for 20 min at room temperature. After this digestion, they were rinsed successively in glycine buffer, PBS and then dehydrated in ethanol. Actual prehybridization was done with 50% formamide, 0.2% sodium dodecyl sulfate (SDS), 0.1% Sarcosyl, 5×standard sodium citrate (SSC; NaCl (0.15M), sodium citrate (0.015M, pH 7.0)) and 2% blocking reagent (Roche Molecular Biochemicals) for 1 h at 60° C. Hybridization was performed by adding the probe (200 ng/ml) to 51 μl of 4×SSC and 50% formamide per section. The slides were incubated overnight at 60° C. in a chamber humidified with 4×SSC and 50% formamide. During hybridization, a coverslip was placed over the tissue section. After hybridization, it was removed and the sections rinsed with 4×SSC, then washed with 4×SSC for 15 min and in 2×SSC for 15 min, at room temperature. Finally, the sections were washed with 0.1% SSC for 30 min. at 60° C. Hybridization was detected by color reaction. For coloration, the sections were washed with Buffers 1 and 2 of the DIG Luminescent Detection Kit (Roche Molecular Biochemicals). They were then incubated with anti-DIG alkaline phosphatase antibody (1:500) in Buffer 2 for 40 min, washed twice in Buffer 1 for 15 min and in Buffer 3 for 2 min. Incubation in the color solution (NBT/x-phos) was carried out for 45 min, after which the slides were washed in distilled water and dry mounted with Geltol.

### In vitro Translation

The full length of the HCaRG coding sequence was synthesized by RT-PCR With specific primers and inserted downstream of the T7 promoter into the pSP72 vector. In vitro transcription and translation were performed using a TNT-T7-coupled reticulocyte lysate system (Promega) in the presence of 35 S-methionine. A plasmid containing the luciferase gene supplied by the manufacturer was used as a control. The synthesized proteins were analyzed by 15% SDS polyacrylamide gel electrophoresis (PAGE) in the absence or presence of β-mercaptoethanol. Radioactive protein bands were detected by scanning with a PhosphorImager.

### Antibody Production

E. coli cells transformed with pGEX-3X were grown in LB medium containing 50 μg/ml ampicillin at 37° C. until A595 nm=0.5 Isopropyl-D-thiogalactopyranoside was added to a final concentration of 0.1 mM, and the cells were cultured for 2 h. Purification of GST-HCaRG was performed according to the manufacturers protocol. Polyclonal antisera with antibodies recognizing HCaRG were produced by immunization of rabbits with GST-HCaRG protein.

### Immunocytological Reaction at the Electron Microscopic Level

Rat tissues (liver, anterior pituitary, spleen, heart and adrenal gland) were quickly removed and fixed in 4% paraformaldehyde with 0.05% glutaraldehyde in phosphate buffer solution for 90 min. A part of the specimens was cryoprotected in 0.4M sucrose phosphate buffer solution for 30 mm at 4° C., then frozen in a cold gradient of fuming nitrogen (Biogel, CFPO, Saint Priest, France) to −4° C., and immersed in liquid nitrogen, as described previously (22). Ultrathin frozen sections of 80 nm thickness were obtained using a dry sectioning method at −120° C. with an Ultracut S microtome (Leica, Lyon, France). The other part of the specimens was dehydrated before embedding in Lowicryl K4M with the AFS system (Leica) (23). Sections were mounted on 400 mesh collodion-carbon-coated nickel grids. For ultrastructural localization of HCaRG protein, the grids were first placed in buffer containing 0.1 M phosphate buffer, 0.15 M NaCl, and 1% albumin, pH 7.4, for 10 min. They were then incubated for 1 h with polyclonal IgG raised against HCaRG protein at concentrations of 1:1000 and 1:50 for ultrathin frozen sections and Lowicryl sections respectively. After 10-min washing in the same buffer antigen/antibody complexes were revealed with anti-rabbit IgG conjugated with 10 nm gold particles in buffer containing 0.05 M Tris, 0.15 M NaCl, 1% albumin, pH 7.6, for 1 h. The grids were washed in the same buffer and fixed with 2.5% glutaraldehyde. The specificity of the immunocytological reaction was tested on sections with omission of primary antibody and incubation of the primary antibody with particle-adsorbed antigen. No signal was observed on these tissue sections. Before observation in a Philips CM 120 electron microscope at 80 kV, the cryosections were contrasted in 2% uranyl acetate, embedded in 8% methylcellulose, and the Lowicryl sections were contrasted for 20 min in 5% uranyl acetate.

### Transfection and Subcellular Localization

COS7 cells were plated at −30-50% confluency 1 day prior to transfection which was performed with 5 μg/well of pEGP-HCaRG or pcDNA1/Neo-HCaRG, according to the calcium phosphate method. After 24 h, the cells were fixed with 4% paraformaldehyde in PBS for 30 min at room temperature. Following 3 washes with PBS, cells transfected with pEGFP-HCaRG or pcDNA1/Neo-HCaRG were mounted on coverslips. The cells were permeabilized with 0.3% Triton X-100 for 12 min, blocked with 1% BSA-1% gelatin for 15 min, incubated with HCaRG antibodies at 37° C. for 1 h, washed in 0.5% BSA, incubated with anti-rabbit FITC-labeled antibodies and washed again. Fluorescence and immunofluorescence were detected with a Zeiss fluorescence microscope.

### Stable Transfection

HEK293 cells were plated in a 100-mm plate at a density of 0.5×10 6 cells/plate. They were transfected with the control plasmid pcDNA1/Neo (Invitrogen, Faraday, U.S.A.) or with the plasmid containing rat HCaRG using a standard calcium phosphate coprecipitation method. 48 h after transfection, the cells were plated in 150-mm plates in the presence of 400 μg/ml G418 (Life Technologies, Burlington, Canada). After 2 weeks, the clones were picked and the level of ectopic HCaRG expression was determined by northern hybridization.

### Cell Counting and 3 H-thymidine Incorporation

The rate of stable clone cell proliferation was measured by counting the number of cells after plating. Cells were seeded at a density of 0.1×10 6 cells/B-well plate, with triplicate plates for each cell line. Every 24 h, the cells were trypsinized and counted in a hemocytometer. HEK293 cells which stably expressed either Neo control plasmid or HCaRG were used for the estimation of DNA synthesis by 3 H-thymidine incorporation. The clones were trypsinised at 90% confluency, counted in a standard hemocytometer and inoculated at an identical initial cell density of 40,000 cells/ml in DMEM containing 10% FBS and G418 at 400 μg/ml. All cells were inoculated in Poly-D-lysine-pretreated 24-well plates in a volume of 1 ml/well (40,000 cells/well). They were allowed to attach and grow for a period of 24-48 h. The growth media were then replaced by DMEM containing 0.2% FBS and G418 (400 μg/ml) for a period of 48 h to synchronise the cells After the synchronisation period, the cells were supplied with fresh medium containing 10% FBS and allowed to grow for 48 h. [³H]-thymidine, 1 μCi/ml (ICN) was added to the cells for the last 4 h of the 48 h-growth period. At the end of incubation, the medium was removed and the monolayers washed twice with PES. The cells were then fixed with ethanol:acetic acid (3:1, V:V), and DNA was digested/extracted with 0.5N PCA at 80-90° C. for 20 min.

The above results show that modulation of the expression of HCaRG has at least an effect on cell proliferation. Overexpression of HCaRG gene leads to inhibition of cell proliferation. This effect of overexpressing the gene (which could be replaced by administering the protein itself) indicates that the gene or the protein, peptide or mimetics are useful at least against proliferative diseases such as cancer. As well, since vascular cells express HCaRG, having these cells to overexpress the gene (or alternatively putting the cells in contact with the HCaRG protein, peptide or mimetic) would reduce cell proliferation provoked by different stimuli (such as occurring during restenosis or artherosclerosis, for example). Of course, any condition where cell proliferation would need to be incrased would be treated the opposite way e.g. by silencing the HCaRG gene or by inhibiting the activity of the gene product.

Another immediate use for the probes or primers capable of hybridizing with HCaRG gene or for the antibodies capable of binding the HCaRG protein is the detection of a Ca-dependent condition High levels are associated to low calcemia while low levels are associated with high calcemia and high calcium-dependent disorders. As shown above, certain types of hypertension as well as hypocalcemia correlates with high levels of HCaRG, while a “high calcium” disease like cancer correlate with low levels of the same.

Although the present invention has been described hereinabove by way of preferred embodiments thereof and annexed figures, it can be modified, without departing from the spirit and nature of the subject invention. Any such modification is under the scope of this invention as defined in the appended claims.

### REFERENCES

1. Brown, E. M., Gamba, G., Riccardi, D., Lombardi, M., Butters, R., Kifor, O. Sun, A., Hediger, M. A., Lytton, J., and Hebert, S. C. (1993) Nature 366, 575-580.

2. Kifor, O., Diaz, R., Butters, R., and Brown, E. M. (1997) J Bone Mineral Res 12, 715-725.

3. Arthur, J. M., Collinsworth. G. P., Getts, T. W., Quarles, L. D., and Raymond, J. R. (1997) Am J Physiol 273, F129-F135.

4. Onyango. I., Hjaim, G., Larsson, M., and Rask, L. (1999) Exp Cell Res 247. 9-16.

5. Kifor, O., Kifor, I., and Brown, E. M. (1992) J Bone Mineral Res 7, 1327-1336.

6. B. Brown, E. M. (1991) Physiol Rev 71, 371-411.

7. Cohn, D. V., Fasciotto, B. H., Zhang. J. X., and Gorr, S. U. (1994) Chemistry and biology of chromogranin A (secretory protein-1) of the parathyroid and other endocrine glands, in J. P. Bilezikian, R. Marcus, and M. A. Levine (eds.), The Parathyroids: Basic and clinical concepts. Raven Press, New York. pp.107-119.

8. Benishin, C. G., Lewanczuk. R. Z., and Pang, P. K. T. (1991) Proc Natl Acad Sci USA 88, 6372-6376.

9. Benishin, C. G., Lewanczuk, R. Z., Shan, J. J., and Pang, P. K. (1999) Am J Hypertens 12, 1260-1263.

10. Sowers, J. R., Zemel, M. B., Standley, P. R., and Zemel, P. C. (1989) J Lab Clin Med 114, 338-348.

11. Resnick. L. M. (1987) Am J Med 82(suppl.1B), 16-25.

12. Resnick. L. M., Laragh, J. H., Sealey, J. E., and Alderman, M. H. (1983) N Engl J Med 309, 888-191.

13. Erne, P. and Hermsmeyer, K. (1989) Hypertension 14,145-151.

14. Feinleib, M., Lenfant, C, and Miller, S. A. (1984) Science 226, 384-386.

15. Drueke, T. B., Chabanis, S., Roullet, C., Duchambon, P., LaCour, B., and McCarron, D. A. (1994) Am J Med Sci 307(Suppl.1), S89-S101.

16. Cirillo, M., Galletti, F., Strazzullo, P., Torielli, L., and Melloni, M. C. (1989) Am J Hypertens 2, 741-746.

17. Orlov, S. N.. Li, J. M., Tremblay, J., and Hamet, P. (1995) Semin Nephrol 15, 669-592.

18. Resnick, L. M. (1995) in Hypertension: Pathophysiology, Diagnosis, and Management (2nd Ed.) (Laragh, J. H. and Brenner, B. M., eds) pp. 119-1619, Raven Press, New York.

19. Kunes, J., Jelinek, J., and Zicha, J. (1986) Clin Sci 70, 185-189.

20. Chomczynsky, P. and Sacchi, N. (1987) Anal Biochem 162, 156-159.

21. Frohman, M. A., Dush, M. K., and Martin, G. R. (1988) Proc Natl Acad Sci USA 85, 8998-9002.

22. Lobie, P. E., Sadir, R,. Graichen, R., Mertani, H. C., and Morel, G. (1999) Exp Cell Res 246,47-55.

23. Gomez, E., Morel, G., Cavalier, A., Lienard, M. O., Haour, F., Courtens, J. L, and Jegou, D. (1997) Biol Reprod 56, 1513-1526.

24. Hatton, D. C. and McCarron, D. A. (1994) Hypertension 23, 513-530.

25. Wilin, A., Zhang, G., Jone, T. W., Jaken, S., and Steven, J. L. (1992) Lab Invest 66, 474-484.

26. Bacallao, R. (1989) Am J Physiol 257, F913-F924.

27. Molitoris, B. A, Hoilien, C. A., Dahl, R., Ahnen, D. J., Wilson, P. D., and Kim, J. (1988) J Membr Biol 106, 233-242.

28. Fried, T. A., Hishida, A., Barnes, J. L., and Stein, J. H. (1984) Am J Physiol 247, F568-F574.

29. Kato. A., Hishida, A., Tanaka, I., and Komatsu, K (1997) Nephron 75, 72-75.

30. Kato, A., Hishida, A., and Nakajima, T. (199) Kidney int 48, 1577-1583.

31. Nakajima, T., Miyaji, T., Kato. A., Ikegaya, N., Yamamoto, T., and Hishida, A. (1996) Am J Physiol 271, F846-F863.

32. Riccardi, D., Park, J., Lee, W. S., Gamba, G., Brown, S. C., and Hebert, S. C. (1995) Proc Natl Acad Sci USA 92, 131-135.

33. Ruat, M., Molliver, M. E., Snowman, A. M., and Snyder, S. H. (1995) Proc Natl Acad Sci USA 92. 3161-3165.

34. Brown, E. M. and Hebert, S. C. (1996) Am J Med Sci 312, 99-109.

35. Garnett, J. E., Capuano, I. V., Hammerland, L. G., Hung, B. C., Brown, E. M., Hebert, S. C., Nemeth, E. F., and Fuller, F. (1995) J Biol Chem 270, 12919-12925.

36. Nygren, P., Gylfe, E., Larsson, R., Johansson, H., Juhlin, C., Klareskoq, L., Akerstrom, G., and Rastad, J. (1988) Biochem Biophys Acta 968, 253-260.

37. Okazaki, T., Ando, K., Igarishi. T., Ogata, T., and Fujita, T. (1992) J Clin Invest 89, 1268-1273

38. McHaffie, G. S. and Ralston, S. H. (1995) Bone 17, 11-14.

39. Reid, R. E Gariepy, J., Saund, A. K, and Hodges, R. S. (1981) J Biol Chem 256, 2742-2751.

40. Kohn, W. D., Mant. C. T., and Hodges, R. S. (1997) J Biol Chem 272, 2583-2586.

41. Shaw, G. S., Hodges, R. S., and Sykes, B. D. (1992) Biochemistry 31, 9572-9580.

42. Kay, L. E., Forman-Kay, J. D., McCubbin, W. D., and Kay, C. M. (1991) Biochemistry 30, 4324-4333.

43. Shaw, G. S., Hodges, R. S. Kay, C. M., and Sykes, B. D. (1994) Protein Sci 3, 1010-1019.

44. Franz, C., Durussel, I., Cox, J. A., Schafer, S. W., and Heizmann, C. W. (1998) J Biol Chem 273, 18826-18834.

45. Linse, S., Thulin, E., and Sellers, P. (1993) Protein Sci 2, 985-1000.

46. Wicki, R., Franz, C., Scholl, F. A., Heizmann, C. W., and Schafer, B. W. (1997) Cell Calcium 22, 243-254.

47. Mueller, A., Bächi. T., Höchi, M., Schäfer, B. W., and Heizmann, C. W. (1999) Histochem Cell Biol 111, 453-459.

48. McKusick, V. A., Francomano, C. A., Antonarakis, S. E., and Pearson, P. L. (1994) Mendelian inheritance in man; A catalog of human genes and genetic disorders., Johns Hopkins. Baltimore, Md.

49. Knuutila, S., Bjorkqvist, A. M., Autio, K., Tarkkanen, M., Wolf, M., Monni, O., Szymanska, J., Larramendy, M. L., Tapper, J., Pere, H., El-Rifai, W., Hemmer, S., Wasenius, V. M., Vidgren, V, and Zhu, Y. (1998) Am J Pathol 152, 1107-1123.

50. Torchia. J., Rose, D. W., Inostroza, J., Kamei, Y., Westin, S., Gilss, C. K., and Rosenfeld, M. G. (1997) Nature 387, 6778.

51. Heery, D. M., Kalkhoven, E., Hoare. S., and Parker, M. G. (1997) Nature 387, 733-736.

52. Huang, Z., Philippin, B., O'Leary, E., Bonventre, J. V., Kriz, W., and Witzgall, R. (1999) J Biol Chem 274, 7640-7648.

53. Witzgall, R., O'Leary, F., Leaf, A., Onaldi, D., and Bonventre, J. V. (1994) Proc Natl Acad Sci USA 91, 4514-4518.

54. Bonventre, J. V. and Witzgall, R. (1996) Exp Nephrol 3, 150-164.

55. Witzgail, R., O'Leary, E., Gessner, R., Ouellette, A. J., and Bonventre, J. V. (1993) Mol Cell Biol 13, 1933-1942.

56. Brown, E. M (1994). Homeostatic mechanisms regulating extracellular and intracellular calcium metabolism, in J. P. Bilezikian, R. Marcus, and M. A. Levine (eds.), The Parathyroids: Basic and clinical concepts. Raven Press, New York, pp-14-54.

57. Pang, P. K. T. and Lewanczuk, R. Z., (1989) Am. J. Hypertens 2, 898-902.

58. Huang, S. L., Wen, Y. I., Kupranycz, D. B., Pang, S. C., Schlager, G., Hamet, P., and Tremblay, J., (1988) J. Clin Invest 82, 276-281.

59. Lewanczuk, R. Z. and P. K. T. Pang, (1989) Clin Exp Hypertens (A) 11, 1471-1485.

60. Chang, E., Kunes, J., Hamet, P., Tremblay, J., (1990) Am J Hypertens 3, 210S-215S.

61. Dash, P. I., Lotan, I., Knapp, M., Kandel, E. R., Goelet, P., (1989) Proc Natl Acad Sci USA 84, 7896.

62. Temblay, J., (1988) J Clin Invest 82, 276-281.

63. Loke, S. L., Stein, C. A., Zhang, X. H., Mori, K., Nakanishi, M., Subasinghe, C., Cohen, J. S., and Neckers, L. M., (1989) Pro Natl Acad Sci USA 86, 3474.

64. Sambrook, J., Fritschand, E. F., Maniatis, T., (1989) Plasmid vector, in J. Sambrook, E. Fritsch, F., and Maniatis, T. (eds.), Molecular cloning: A laboratory manual. Cold Spring Harbor Laboratory Press, New York, pp.1.1-1.110,

65. Stein, C A, and Cohen, J. S., (1988a) Cancer 48, 2659.

66. Stein, C A, Mori, K, Loke, S. L., Subasinghe, C., Shinazuka, K., Cohen, J. S., and Meckers, L. M (1988b) Gene 72, 333.

67. Tremblay, J., Chang, E., Kunes, J., and Hamet P. (1991) Clin Exp Hypertens (A) 13. 735.

68. McKenzie, J. C., Scot@ J. N., and Inagami, T. (1991) Am.J.Anat 190,182-191

69. Figueroa, C. D., Lewis, H. M., Maciver, A. G., Mackenzie, J. C., and Bhoola, K. D. (1990) Nephrol.Dial.Transplant 5,26-31

70. Greenwald, J. E., Needleman, P., Wilkins, M. R., and Schreiner, G. F. (1991) Am. J. PhysioL 260, F602-F607 18. Rifter, D., Needleman, P., and Greenwald, J. E. (1991) J.Clin.Invest 87,208-212

71. Lai, K. N., Leung, J. C., Yandle, T. G., Fisher, S., and Nicholls, M. G. (1999) J.Hypertens 17, 575-583

72. Lenz, W., Herten, M., Gerzer, R., and Drummer, G. (1999) Kidney Int 55, 91-99

What is claimed is:

1. A nucleic acid molecule isolable from parathyroid of a mammal and whose expression is regulated by extracellular calcium concentration, or its complementary strand.

2. A nucleic acid molecule as defined in claim 1, having the sequence set out in SEQ ID No. 1, or its complementary strand.

3. A nucleic acid molecule, having an homology of at least 60% with the nucleic acid of claim 2.

4. A nucleic acid molecule, having an homology of at least 80% with the nucleic acid of claim 2.

5. A nucleic acid molecule as defined in claim 4, wherein the mammal is a human.

6. A nucleic acid as defined in claim 5, which has the sequence set out in SEQ ID No. 3.

7. A recombinant vector comprising the nucleic acid of claim 1.

8. A recombinant vector comprising the nucleic acid of claim 5.

9. A recombinant host cell comprising the recombinant vector of claim 7.

10. A recombinant host cell comprising the recombinant vector of claim 8.

11. A nucleic acid of at least 12 nucleotides in length capable of a specific hybridization with the nucleic acids of a calcium sensing cell and with SEQ ID No. 1, or SEQ ID No. 3, or a complementary sequence thereof.

12. A nucleic acid as defined in claim 11 which is an amplification primer.

13. A nucleic acid as defined in claim 11, which is a hybridization probe.

14. A composition of matter comprising the nucleic acid of claim 1 and a carrier.

15. A composition of matter comprising the nucleic acid of claim 5 and a carrier.

16. A composition of matter comprising the nucleic acid of claim 6 and a carrier.

17. A composition of matter comprising the recombinant vector of claim 7 and a carrier.

18. A composition of matter comprising the recombinant vector of claim 8 and a carrier.

19. A composition of matter comprising the nucleic acid of claim 11 and a carrier.

20. A composition of matter comprising the nucleic acid of claim 12 and a carrier.

21. A composition of matter comprising the nucleic acid of claim 13 and a carrier.

22. A composition of matter comprising the recombinant host cell of claim 9 and a carrier.

23. A composition of matter comprising the recombinant host cell of claim 10 and a carrier.


SEQUENCE LISTING (4 sequences)

SEQ ID NO: 1 - DNA, 1100 bp, Rattus rattus
  CDS (132)..(803)
gcacgagccacagccagctaccgcggctaggttcctccaggtgcagagggcggtaaaggcttggtttgtatttgtaatgcaactgtggttaggaccttctcttcggactggtcaagaaacgggaagaaaggatgtctgctttgggggctgcagctccatacttgcaccatcccgctgacagtcacagtggccgggtcagtttcctgggttcccagccctctccagaagtgacggccgtggctcagctcttgaaggacttagacaggagcaccttcagaaagttgttgaaacttgtagtcggggccctgcatgggaaagactgcagagaagctgtggagcaacttggtgccagcgccaacctgtcagaagagcgtctggccgtcctgctggcgggcacacacaccctgctccagcaggctctccggctgccccctgctagtctaaagccagatgccttccaggaagagctccaggaacttggcattcctcaggatctaattggagatttggccagtttggcatttgggagtcaacgccctcttctcgactctgtagcccaacagcagggatcctcgctgcctcacgtgtcttacttccggtggcgggtggacgtggccatctcaaccagcgctcagtcccgctccctgcaaccgagtgttctcatgcagctgaagctcacagatggatctgcacaccgcttcgaggtgcccatagccaaatttcaggagctgcggtacagtgtagccttggtccttaaggagatggcagaactggagaagaagtgtgagcgcaaactgcaggactgactgaaccctggtactgtgggtgctgaagctggtaccagaacacagccccccactggtgatgagcccaactccattgaggtcctgcatgtgagaacgtattttaagtgaaaagacagcgggactttcaggttttgttttatgagtcaacagctgggcagggtggcacagtttataatctcagcccttggaagtctgaggctggagaatgggaagtgtaagctgggcctggctttcatagtgaggctcagtgtcgaattaaagaggtaaagcaactattaaaaaaaaaaaaaaaaa

SEQ ID NO: 2 - protein, 224 aa, Rattus rattus
MSALGAAAPYLHHPADSHSGRVSFLGSQPSPEVTAVAQLLKDLDRSTFRKLLKLVVGALHGKDCREAVEQLGASANLSEERLAVLLAGTHTLLQQALRLPPASLKPDAFQEELQELGIPQDLIGDLASLAFGSQRPLLDSVAQQQGSSLPHVSYFRWRVDVAISTSAQSRSLQPSVLMQLKLTDGSAHRFEVPIAKFQELRYSVALVLKEMAELEKKCERKLQD

SEQ ID NO: 3 - DNA, 1355 bp, Homo sapiens
  CDS (295)..(966)
gggcaggcagttgaggtggattaaaccaaacccagctacgcaaaatcttagcatactcctcaattacccacataggatgaataatagcagttctaccgtacaaccccggaggcgcagaccgtccacacgggacagggacgcccgctcggctccgctccgcgctgatcctcaaggtctcgcggtttccccgccgcccacccggacgccgacgaaagccagcgagctcctcagcctcaggcatctgcatctgggaccgacctcctgggctggctgatcaaagaggaagcagcagcaatgtctgctgtgggggctgcaactccatacctgcatcatcctggtgatagtcacagtggccgagtgagtttcttgggggcccagcttcctccagaggtggcagcaatggcccggctactaggggacctagacaggagcacgttcagaaagttgctgaagtttgtggtcagcagcctgcagggggaggactgccgagacggtgtgcagcgtcttggggtcagcgccaacctgccggaggagcagctgggtgccctgctggcaggcatgcacacactgctccagcaggccctccgtctgccccccaccagcctgaagcctgacaccttcagggaccagctccaggagctctgcatcccccaagacctggtcggggacttggccagcgtggtatttgggagccagcggcccctccttgattctgtggcccagcagcagggggcctggctgccgcatgttgctgactttcggtggcgggtggatgtagcaatctccaccagtgccctggctcgctccctgcagccgagcgtcctgatgcagctgaagctttcagatgggtcagcataccgctttgaggtccccacagccaagttccaggagctgcggtacagcgtggccctggtcctaaaggagatggcagatctggagaagaggtgtgagcgcagactgcaggactgacccctcacttgaccagtcccattcagatccggcttggacaggcacctgagatggtgccaaagtgcagctgactcttcccacgacagccctggccttcccatcaggcaggctcttcagtgagtgtttgaacgtaattatgtagttttctgtttaattgaaaaagagagctatgcctttttttctttttggaagtaaagcagctaaaacatgtttctataggtgagtgttggaccttcacacctccccttccctgtacatttgtctttggtgctggacgtggccatgtgaggccaggttgaggccctttgtagacaacatacagttgctcagcctggccccatgtagccaggtgcttttgtagatcttgtgtttcaggcagggcccgg

SEQ ID NO: 4 - protein, 224 aa, Homo sapiens
MSAVGAATPYLHHPGDSHSGRVSFLGAQLPPEVAAMARLLGDLDRSTFRKLLKFVVSSLQGEDCRDGVQRLGVSANLPEEQLGALLAGMHTLLQQALRLPPTSLKPDTFRDQLQELCIPQDLVGDLASVVFGSQRPLLDSVAQQQGAWLPHVADFRWRVDVAISTSALARSLQPSVLMQLKLSDGSAYRFEVPTAKFQELRYSVALVLKEMADLEKRCERRLQD